SEQUENCE LISTING (24 sequences)

SEQ ID NO: 1 - DNA, 50 bp, Artificial Sequence
  Description of Artificial Sequence Synthetic oligonucleotide
gcttcacaaccgcacttgatttaatagaccataccgtctattatttctgg

SEQ ID NO: 2 - DNA, 26 bp, Artificial Sequence
  Description of Artificial Sequence Synthetic oligonucleotide
tttaactttaagaaggagatatacat

SEQ ID NO: 3 - DNA, 714 bp, Artificial Sequence
  Description of Artificial Sequence Synthetic polynucleotide
atgcgtaaaggtgaagaactgttcaccggtgttgttccgatcctggttgaactggacggtgacgttaacggtcacaaattctctgttcgtggtgaaggtgaaggtgacgctaccaacggtaaactgaccctgaaattcatctgcaccaccggtaaactgccggttccgtggccgaccctggttaccaccctgacctacggtgttcagtgcttcgctcgttacccggaccacatgaaacagcacgacttcttcaaatctgctatgccggaaggttacgttcaggaacgtaccatctctttcaaagacgacggtacctacaaaacccgtgctgaagttaaattcgaaggtgacaccctggttaaccgtatcgaactgaaaggtatcgacttcaaagaagacggtaacatcctgggtcacaaactggaatacaacttcaactctcacaacgtttacatcaccgctgacaaacagaaaaacggtatcaaagctaacttcaaaatccgtcacaacgttgaagacggttctgttcagctggctgaccactaccagcagaacaccccgatcggtgacggtccggttctgctgccggacaaccactacctgtctacccagtctgttctgtctaaagacccgaacgaaaaacgtgaccacatggttctgctggaattcgttaccgctgctggtatcacccacggtatggacgaactgtacaaa

SEQ ID NO: 4 - DNA, 33 bp, Artificial Sequence
  Description of Artificial Sequence Synthetic oligonucleotide
aattaatacgactcactatagggagaccacaac

SEQ ID NO: 5 - DNA, 660 bp, Artificial Sequence
  Description of Artificial Sequence Synthetic polynucleotide
atgccgctgaccgacaccccgccgtctgttccgcagaaaccgcgtcgtggtcgtccgcgtggtgctccggacgcttctctggctcaccagtctctgatccgtgctggtctggaacacctgaccgaaaaaggttactcttctgttggtgttgacgaaatcctgaaagctgctcgtgttccgaaaggttctttctaccactacttccgtaacaaagctgacttcggtctggctctgatcgaagcttacgacacctacttcgctcgtctcctcgaccaggcgttcctggacggttcgctggctccgctggctcgtctgcgtctgttcacccgtatggctgaagaaggtatggctcgtcacggtttccgtcgtggttgcctggttggtaacctgggtcaggaaatgggtgctctgccggacgacttccgtgctgctctgatcggtgttctggaaacctggcagcgtcgtaccgctcagctgttccgtgaagctcaggcttgcggtgaactgtctgctgaccacgacccggacgctctggctgaagctttctggatcggttgggaaggtgctatcctgcgtgctaaactggaactgcgtccggacccgctgcactctttcacccgtaccttcggtcgtcacttcgttacccgtacccaggaataa

SEQ ID NO: 6 - DNA, 41 bp, Artificial Sequence
  Description of Artificial Sequence Synthetic oligonucleotide
aaccccttggggcctctaaacgggtcttgaggggttttttg

SEQ ID NO: 7 - DNA, 52 bp, Artificial Sequence
  Description of Artificial Sequence Synthetic primer
aattaatacgactcactatagggagaccacaacatgccgctgaccgacaccc

SEQ ID NO: 8 - DNA, 63 bp, Artificial Sequence
  Description of Artificial Sequence Synthetic primer
caaaaaacccctcaagacccgtttagaggccccaaggggttttattcctgggtacgggtaacg

SEQ ID NO: 9 - DNA, 4200 bp, Artificial Sequence
  Description of Artificial Sequence Synthetic polynucleotide
atgatcgataccgcaccgctggcaccgccgcgtgctccgcgcagcaatccgattcgtgatcgcgtggattgggaagcgcagcgtgcagcagcactggccgatccgggtgcatttcatggtgcgatcgcccgtaccgttattcactggtatgatccgcagcatcactgctggattcgcttcaacgaaagctctcagcgttgggaaggtctggatgcagcaacgggtgctccggttacagtggattatcctgccgattaccagccgtggcagcaggcatttgatgatagtgaagcgccgttttatcgctggttcagcggcggtctgacgaacgcatgttttaatgaagttgatcgtcacgtgacaatgggttacggcgatgaagtggcgtattacttcgaaggtgatcgctgggataatagcctgaacaatggccgtggcggtccggtggttcaggaaacgattacccgtcgccgtctgctggttgaagtggttaaagcagcgcaggttctgcgcgatctgggcctgaaaaaaggtgatcgtatcgcgctgaacatgccgaatatcatgccgcagatttattacaccgaagccgcaaaacgcctgggtattctgtatacgccggtgtttggcggtttcagtgataaaaccctgagcgatcgcatccataatgcaggtgcgcgtgtggttattacctctgatggcgcgtatcgtaacgcccaggtggttccgtataaagaagcctacacggatcaggcactggataaatacatcccggtggaaaccgcccaggcaattgttgcacagacgctggcaaccctgccgctgaccgaaagtcagcgccagacgattatcaccgaagtggaagcagcactggcaggtgaaattacggttgaacgttctgatgttatgcgcggtgtgggcagtgcgctggccaaactgcgcgatctggatgccagtgtgcaggcaaaagttcgtaccgtgctggcacaggcgctggttgaaagcccgccgcgcgtggaagcagtggttgtggttcgtcatacgggtcaggaaatcctgtggaatgaaggccgtgatcgctggagccacgatctgctggatgcagcactggcgaaaattctggctaacgcacgcgccgcaggttttgatgttcactctgaaaacgatctgctgaatctgccggatgatcagctgatccgtgctctgtatgcgagtattccgtgcgaaccagttgatgccgaatatccgatgtttattatctacacgagcggttctaccggcaaaccgaaaggtgttattcatgttcacggcggttacgtggcgggcgtggttcataccctgcgcgttagtttcgatgccgaaccgggcgatacgatttatgtgatcgcagatccgggctggatcacaggtcagagctacatgctgacggcaaccatggcaggtcgtctgactggtgtgattgccgaaggttctccgctgtttccgagtgcgggccgctatgcctctattatcgaacgttacggtgttcagatttttaaagcgggcgttacgttcctgaaaaccgtgatgagtaacccgcagaatgttgaagatgtgcgcctgtatgatatgcacagtctgcgtgtggcaaccttttgtgcagagccggttagcccggcagtgcagcagttcggtatgcagatcatgacgccgcagtatattaatagctactgggcgacggaacatggcggtattgtgtggacccacttttatggcaaccaggatttcccgctgcgtccagatgcacatacgtacccgctgccgtgggttatgggtgatgtttgggtggcagaaaccgatgaatctggcaccacgcgctatcgcgtggcggatttcgatgaaaaaggtgaaatcgttatcaccgcaccgtatccgtacctgacgcgaaccctgtggggtgatgtgccgggttttgaagcgtatctgcgtggtgaaatcccgctgcgtgcatggaaaggtgatgcagaacgtttcgttaaaacctactggcgtcgtggtccgaatggcgaatggggttatatccagggcgattttgcgattaaatacccggatggtagtttcacgctgcatggccgcagcgatgatgttattaatgtgtccggccaccgtatgggtacggaagaaatcgaaggtgccattctgcgtgatcgccagatcaccccggattctccggtgggtaactgcattgtggttggcgcgccgcatcgtgaaaaaggcctgaccccggttgcatttatccagccagcaccgggtcgtcacctgacgggtgcagatcgccgtcgcctggatgaactggtgcgtaccgaaaaaggtgcagttagcgtgccggaagattatattgaagttagtgcgtttccggaaacccgcagcggtaaatacatgcgtcgcttcctgcgtaatatgatgctggatgaaccgctgggcgataccacgaccctgcgcaacccggaagtgctggaagaaatcgcggccaaaattgccgaatggaaacgtcgccagcgcatggcagaagaacagcagattatcgaacgttatcgctactttcgtattgaatatcatccgccgaccgcaagtgcaggtaaactggcagtggttacggttaccaatccgccggtgaacgccctgaatgaacgtgctctggatgaactgaacaccatcgtggatcacctggcgcgtcgccaggatgttgcagcgattgtgtttacgggtcagggtgctcgcagcttcgtggccggtgcggatatccgtcagctgctggaagaaattcataccgttgaagaagccatggcactgccgaacaatgcgcacctggcctttcgcaaaattgaacgtatgaacaaaccgtgcattgccgcaatcaatggtgtggcactgggcggtggcctggaatttgcgatggcctgtcattatcgcgttgccgatgtgtacgcagaatttggtcagccggaaatcaacctgcgtctgctgccgggttatggtggtacgcagcgtctgccgcgtctgctgtacaaacgcaacaatggtacaggcctgctgcgtgcgctggaaatgattctgggtggccgcagcgtgccagcagatgaagcactggaactgggtctgattgatgcaatcgcgaccggcgatcaggatagtctgagcctggcctgcgcactggcgcgtgcggcaatcggtgcagatggtcagctgattgaaagcgcagcggtgacccaggcctttcgtcatcgccacgaacagctggatgaatggcgtaaaccggacccgcgcttcgcggatgatgaactgcgctctattatcgcccatccgcgtatcgaacgcattatccgtcaggcgcataccgttggtcgtgatgcagcagtgcaccgtgcactggatgcaattcgttatggcattatccatggttttgaagccggcctggaacacgaagcaaaactgttcgccgaagcagtggttgatccgaatggtggcaaacgcggcatccgtgaatttctggatcgtcagtctgcaccgctgccgacacgtcgcccgctgattaccccggaacaggaacagctgctgcgtgatcagaaagaactgctgccggtgggtagtccgtttttccctggcgttgatcgcatcccgaaatggcagtatgcgcaggccgtgattcgtgatcccgatactggtgcagcagcacatggcgatccgatcgttgcggaaaaacagattatcgttccggtggaacgtccgcgtgcgaaccaggcactgatttacgttctggcgagcgaagtgaactttaatgatatttgggccatcacaggtattccggtgagccgcttcgatgaacatgatcgtgattggcacgtgacgggttctggtggcatcggcctgattgttgcgctgggcgaagaagcccgtcgcgaaggtcgtctgaaagttggcgatctggtggcgatctatagcggccagtctgatctgctgagcccgctgatgggtctggacccgatggcagccgattttgtgattcagggtaatgataccccggatggctctcatcagcagttcatgctggcacaggcaccgcagtgcctgccgatcccgacggatatgagcattgaagcagcgggttcttatatcctgaacctgggcaccatttaccgcgcactgtttacgaccctgcaa

SEQ ID NO: 10 - DNA, 320 bp, Artificial Sequence
  Description of Artificial Sequence Synthetic polynucleotide
agaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgctttcatatctttcactttttttgggctaac

SEQ ID NO: 11 - DNA, 879 bp, Artificial Sequence
  Description of Artificial Sequence Synthetic polynucleotide
atggctgaagcgcaaaatgatcccctgctgccgggatactcgtttaacgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgcattagtcaggcgaagctgcttttgagcactacccggatgcctatcgccaccgtcggtcgcaatgttggttttgacgatcaactctatttctcgcgagtatttaaaaaatgcaccggggccagcccgagcgagtttcgtgccggttgtgaagaaaaagtgaatgatgtagccgtcaagttgtcataa

SEQ ID NO: 12 - DNA, 521 bp, Artificial Sequence
  Description of Artificial Sequence Synthetic polynucleotide
atgctgttgattgacgccagtgagaacccggaaccggaaacggaatcaaatccgtgggtcgaacagtggggcacgctgttgtcctgatatgttcagcgagcggtaaatgtcgttttagcggtgctgaatcgaatcttttcaggcaaatgccagtaaaaactgcttcatagcgcggatttttactggcgtttgcctggagtcaagcgatccatttcatactcttctttatttcttcgttttaacccttcctttcttgttcttgttttcatttccgtgaagtggattccaccgtccagggctaatgccaaaatcgggcctcattgaacgcattaatgttgtgttgttgcacggtgagccgctatggcgcgctttttatactgctattgccagatataaacacgcgccgtattcggcgaacgacctataaaaacggcaaaaaacaccctacgtcacctctgatttcctggcgatgtcgcagtccagagtgagcgtggctaacgcgaattttcaggagtgcaaca

SEQ ID NO: 13 - DNA, 1158 bp, Artificial Sequence
  Description of Artificial Sequence Synthetic polynucleotide
atggctggctggcatcttgataccaaaatggcgcaggatatcgtggcacgtaccatgcgcatcatcgataccaatatcaacgtaatggatgcccgtgggcgaattatcggcagcggcgatcgtgagcgtattggtgaattgcacgaaggtgcattgctggtactttcacagggacgagtcgtcgatatcgatgacgcggtagcacgtcatctgcacggtgtgcggcaggggattaatctaccgttacggctggaaggtgaaattgtcggcgtaattggcctgacaggtgaaccagagaatctgcgtaaatatggcgaactggtctgcatgacggctgaaatgatgctggaacagtcgcggttgatgcacttgttggcgcaggatagccgtttgcgggaagaactggtgatgaacctgattcaggcagaggagaatactcccgcacttactgaatgggcgcaacggctggggatcgatctcaatcaaccgcgagtggtggctattgttgaggtcgacagcggtcagcttggcgtggacagcgcaatggcggagttacaacaactgcaaaacgcgctgactacgcccgagcgtaataatctggtggcgattgtctcgctaaccgaaatggtggtgttgaaaccggcgttgaactcttttgggcgctgggatgcagaagatcatcgtaagcgagttgaacaactgattacccgcatgaaagagtacggccagctgcgttttcgcgtttcactgggcaactattttaccggtcctggcagtattgcccgatcctatcgtacggcgaaaacgacgatggtggtgggtaaacagcggatgccagaaagtcgctgctatttttatcaggatctgatgttacctgtgttactcgacagtttgcgtggcgactggcaggccaacgaactggcgcgaccgctggcgcggctgaaaacgatggacaataacggcttgctgcgacgaacgctggcggcgtggtttcgccacaatgtgcaaccgctggcaacgtcaaaggcgttgtttattcatcgtaataccctggagtatcggcttaatcgtatatcggaactgaccgggcttgatttgggcaattttgatgacaggttgctgctgtatgtggcgttacaactggatgaagagcggtag

SEQ ID NO: 14 - DNA, 52 bp, Artificial Sequence
  Description of Artificial Sequence Synthetic oligonucleotide
ggattgaatataaccgacgtgactgttacatttaggtggctaaacccgtcaa

SEQ ID NO: 15 - DNA, 585 bp, Artificial Sequence
  Description of Artificial Sequence Synthetic polynucleotide
atgccgcgtccgaaactgaaatctgacgacgaagttctggaagcggcgaccgttgttctgaaacgttgcggtccgatcgaattcaccctgtctggtgttgcgaaagaagttggtctgtctcgtgcggcgctgatccagcgtttcaccaaccgtgacaccctgctggttcgtatgatggaacgtggtgttgaacaggttcgtcactacctgaacgcgatcccgatcggtgcgggtccgcagggtctgtgggaattcctgcaggttctggttcgttctatgaacacccgtaacgacttctctgttaactacctgatctcttggtacgaactgcaggttccggaactgcgtaccctggcgatccagcgtaaccgtgcggttgttgaaggtatccgtaaacgtctgccgccgggtgcgccggcggcggcggaactgctgctgcactctgttatcgcgggtgcgaccatgcagtgggcggttgacccggacggtgaactggcggaccacgttctggcgcagatcgcggcgatcctgtgcctgatgttcccggaacacgacgacttccagctgctgcaggcgcacgcgtaa

SEQ ID NO: 16 - DNA, 91 bp, Artificial Sequence
  Description of Artificial Sequence Synthetic oligonucleotide
tcgagtccctatcagtgatagagattgacatccctatcagtgatagagatactgagcacatcagcaggacgcactgaccgaattcattaaa

SEQ ID NO: 17 - DNA, 624 bp, Artificial Sequence
  Description of Artificial Sequence Synthetic polynucleotide
atgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaa

SEQ ID NO: 18 - DNA, 51 bp, Artificial Sequence
  Description of Artificial Sequence Synthetic oligonucleotide
cacccagcagtatttacaaacaaccatgaatgtaagtatattccttagcaa

SEQ ID NO: 19 - DNA, 633 bp, Artificial Sequence
  Description of Artificial Sequence Synthetic polynucleotide
atggtgcgtcgcaccaaagaagaagcacaggaaacgcgtgcgcagattatcgaagcggccgaacgcgcgttttataaacgtggtgtggcacgtaccacgctggcagatattgcagaactggcaggtgttacccgcggtgcaatctactggcatttcaacaataaagccgaactggttcaggcactgctggattctctgcacgaaacgcatgatcacctggcccgtgcaagcgaatctgaagatgaactggacccgctgggctgcatgcgcaaactgctgctgcaggtgtttaacgaactggttctggatgcacgtacccgtcgcattaatgaaatcctgcatcacaaatgcgaatttacggatgatatgtgtgaaattcgtcagcagcgccagagcgccgtgctggattgtcataaaggtatcaccctggcactggcaaacgcagttcgtcgcggtcagctgccgggtgaactggatgtggaacgcgcagcggttgcgatgtttgcctatgtggatggcctgattggtcgttggctgctgctgccggatagtgttgatctgctgggcgatgtggaaaaatgggttgataccggtctggatatgctgcgtctgagcccggcgctgcgcaaataa

SEQ ID NO: 20 - DNA, 1023 bp, Candida albicans
atggtaaacaaggtcggtaaatctactctcgataagagcacaaacctagataaatccaaagggaatatattagagaaactagatgatgatatacttcatgtcaatagaattcgaggctctttaactaacaaaactccaatcaccaaaacccattcgatagatgatgagcttaaactagaagaacaatcagaaactgccgccgatgaaaattggcaaatagcatcggaatattataaaaacatagacacgaaggctttccgccaatatgaattagcttgtgatagagtcaaacagttttatgaagaacaacatgaaaaacaaaccgtggcgtataatattcaagcaagaattaatttcaaaactaaaacaagagcaagaatgacagtttgggaaggactagagaaattaaacaaattgttagatgattctgatcccgacaccgaattgtcacaaatagatcatgcattacagacggcagaagctatacggcgagatgggaaaccacgatggtttcaattagttgggttgattcatgatttagggaaattactatatttttttgattctcgtggtcaatgggatgtagtgggtgatactttccctgttggttgtaaattcctgaaacggattattttccctgatagttttaaaaataatccagatttcctaaatccattgtataataccaaatatggcatatattcaaaacattgtggattagataaagtcatgttgagttggggtcatgatgagtatatgtatcatgttgcgaaaaagaattcgacattaccaccggaagcattggcaatgataaggtatcattcattttatccttggcatcaagaattggcatatagttatttaatggatgagcatgataaagagatgttgaaagcagtcaaagctttcaattcctatgatttatattccaagatagatcaacagtatgatgttgaagagttgaaaccatattacctagagttgattgatgagtttttcccaaataaagtaattgatttttaa

SEQ ID NO: 21 - DNA, 978 bp, Francisella sp.
atgagtcagaccgtggaaaacacgtttggcgaatttcgtaactacaccgatagcaaattccaggatcgtgtggaacgcacgtacaaagatatgcacattaaccagaatctggaatacgttacccagatgaaagataaatacttcaaactggatctgggtaaaatggatgtgtacgaagttttcaaactgctggaaaacgttcatgatgaaagcgatccggataatgatctgccgcagatcgaacacgcatatcagaccgcggaagcctgccagaacaaattcctgaaatctgatacggaactgcgcgaaaatgcgctgattcgtagtatctttcgcgatcatgaatggcagagcattccgaaaatctggcaggatttctataccaaaaaacagagtctgggcaatctgtacagccatattaaagattggtcttggtttccgctggttggcttcgttcacgatctgggtaaaatcatgaccctgccggaatatggtcagctgccgcagtggagcaccgtgggtgatacgtacccgattgcctgcccgtttgcaagcgcgaacgtgttttctcaccgtgaatttgttaaagattctaaagattacaacaattacaataccgaaagtgaaacgcattatggcaaatacgagaaaaaatgtggtttcgataacgtggatatgagcttcggtcacgatgaatacatctacaaagttttcgaacagggcagcgatatcccgtatgaaggtctgtacctgctgcgctatcattctttctacccgtggcacaccccgcagacgggcggtcatgcgtatcaggaactggccaacgaaaaagattggctgctgctgccgctgctgaaagcctttcagaaagcggatctgtattctaaactgccggaactgccgccgaaagaagtgctggagaaaaaatacaaaagtctgctggataaatgggttccgaacaagaaaattaactggtaa

SEQ ID NO: 22 - DNA, 888 bp, Flavobacterium johnsoniae
atgaaaaagcatatagacacagacaatccgttgaaaaatttagatgagtgggaagatgatttgttaatgcgatatcctgacccttctgaagtaaatgaaagtttaaaagaaaagcagaaagaagaatttagaaattatgtcgattctgaaagagtagaaacggtaaaagaattttacaggataaaccatacctaccaaacttatgactttgtatgcagtaaagaacaagaatttctgcaatttaatagaaaagaaatgtcaatctgggaagctgtcgagtttttaaacacgcttgtagacgacagtgacccagatattgacttagaccagacacagcaccttttacagacttcagaagccattcgtgctgatggtcatccggattggtttgtactgacaggtttcattcacgatttgggtaaagttttatgcttatttggagaaccgcaatgggcagtcgttggcgatacttttccggttggctgtgcgtattcggataaaattgtgtattcagaattttttaaagaaaatccggattatacagatgagagattcaatactaaactaggaatctacactgaaaactgcggattagataacgtaaaaatgagctggggtcatgacgaatatttgtatcagattatgaaagattatttaccggatcctgctttatacatgattcgttatcactctttttattcgcagcataaagaaaatgcgtatgcacatttaatgaatgaaaaagacatcgaaatgtttgactgggttcgaaaattcaatccgtacgatttgtatacaaaggctcctgtaaaaccagatgttcaggcattacttccttattataaagaattagttgctaaatatttgcctgaaaaattgaagttttaa

SEQ ID NO: 23 - DNA, 858 bp, Mus musculus
atgaaagtggatgttggcccggacccgagcctggtttaccgcccggatgtggacccggaaatggcaaaaagcaaagattcgtttcgtaactacaccagtggcccgctgctggatcgtgtttttaccacgtataaactgatgcatacccaccagacggttgactttgtcagccgtaaacgcattcaatatggcggtttctcttacaagaaaatgaccatcatggaagcggtgggcatgctggatgacctggttgatgaatcagatccggacgtcgattttccgaattcgtttcatgcgttccagacggccgaaggtattcgcaaagcccacccggacaaagattggttccatctggtcggcctgctgcacgatctgggtaaaatcatggcactgtggggtgaaccgcagtgggctgtggttggtgatacctttccggtgggttgccgtccgcaagcaagtgtcgtgttttgtgactccaccttccaggacaacccggatctgcaagacccgcgctattcaacggaactgggcatgtaccagccgcattgcggtctggaaaacgtgctgatgtcgtggggtcacgatgaatacctgtaccagatgatgaaattcaacaaattcagcctgccgtctgaagccttctacatgatccgtttccatagtttctacccgtggcacaccggcggtgattatcgccagctgtgctcccagcaagacctggatatgctgccgtgggtgcaagaattcaacaaattcgatctgtacacgaaatgtccggatctgccggacgttgaatctctgcgtccgtactaccaaggtctgattgataaatactgtccgggcaccctgtcgtggtaa

SEQ ID NO: 24 - DNA, 4813 bp, Artificial Sequence
  Description of Artificial Sequence Synthetic polynucleotide
attcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatataccatgggtcatcaccaccaccatcacggtggcgctagcaaaggtgaagagctgtttacgggtgtagtaccgatcttagtggaattagacggcgacgtgaacggtcacaaatttagcgtgcgcggcgaaggcgaaggtgacgctaccaatggtaaattgaccctgaagtttatttgcacaacaggcaaattacccgttccgtggcccaccttagtgaccaccctgacctatggcgttcagtgcttcagtcgttacccagatcatatgaaacaacacgattttttcaaatcagccatgcctgaaggatatgttcaagagcgtacaatcagcttcaaggacgatggcacctataaaacgcgtgcggaagtgaaatttgaaggcgacacattagtaaaccgtatcgaactgaaaggtatcgacttcaaagaagacggcaacattttaggccataagctggaatataactttaattctcataacgtgtatattacggccgataaacagaaaaacggtatcaaggcaaatttcaaaattcgccataacgtggaagacggcagcgttcaattagcggatcattatcaacaaaacacgccgattggtgacgggcctgtactgttacctgacaaccactacctgagcacccagtcagcactgagcaaagatccgaacgaaaaacgcgatcacatggttctgttagaattcgtgaccgctgcaggcattactcacggaatggacgaactctacaagtaataatgaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgc

(19) United States
(12) Patent Application Publication
(10) Pub. No.: US 2019/0112598 A1
(43) Pub. Date: Apr. 18, 2019

(54) Methods and Systems of Cell-Free Enzyme Discovery and Optimization

(71) Applicant: President and Fellows of Harvard College, Cambridge, MA (US)

(72) Inventors: George M. CHURCH, Brookline, MA (US); Jameson K. ROGERS, Boston, MA (US); Marc GUELL, Barcelona (ES); Alexander GARRUSS, Cambridge, MA (US)

(21) Appl. No.: 16/083,146

(22) PCT Filed: Mar. 7, 2017
(86) PCT No.: PCT/US17/21087

§ 371 Date: Sep. 7, 2018

Related U.S. Application Data
(60) Provisional application No. 62/305,586, filed on Mar. 9, 2016.

(51) Int. Cl. C12N 15/10

(52) U.S. Cl. CPC C12N 15/1086

(57) ABSTRACT

Methods and systems of cell-fee enzyme discovery and optimization are provided.

### RELATED APPLICATION DATA

This application claims priority to U.S. Provisional Application No. 62/305,586 filed on Mar. 9, 2016 which is hereby incorporated herein by reference in its entirety for all purposes.

### STATEMENT OF GOVERNMENT INTERESTS

This invention was made with government support under DE-FG02-02ER63445 awarded by the U.S. Department of Energy. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 2, 2017, is named 010498_00665US_SL.txt and is 29,599 bytes in size.

### FIELD

The present invention relates in general to methods and systems of cell-free enzyme discovery and optimization.

### BACKGROUND

Discovery of new enzymatic systems for the production of valuable chemicals is a slow and expensive process. Enzymatic systems are used to turn readily available precursor compounds into products such as fuels, fine chemicals, pharmaceuticals, agricultural products or even perfumes and fragrances. Enzymatic systems are composed of one or more enzymes that catalyze the necessary series of chemical reactions that take a precursor compound to a product compound. The market for enzyme-catalyzed products currently stands at S150 billion of sales each year and is growing rapidly.

Many times the enzyme necessary for a desired chemical reaction doesn't exist. Other times an enzyme exists but performs sub-optimally. In these situations, it is necessary to make mutations to the enzyme to change its behavior such that it performs at the necessary level. A small enzyme of 300 amino acids can have more variations than there are stars in the universe. If prior knowledge allows an engineer to identify just 5 locations where mutations may be beneficial then the number of possible mutants is reduced to just 100 trillion. Because the number of possible enzyme mutants is so vast and our knowledge of enzyme design so limited the amount of time it takes to find better enzyme variants can be the limiting factor in achieving reasonable product development periods. However, biotechnology firms are strongly motivated to discover new and better enzyme variants because enzyme productivity directly impacts firm profitability.

Because evaluation of enzyme productivity remains the major bottleneck in the discovery of novel and enhanced enzymes this is the most attractive area to innovate in. However, current methods for discovering better enzyme variants rely on low-throughput measurement techniques such as liquid or gas chromatography and mass spectrometry. These primary methods for evaluating enzyme quality require 5-20 minutes of analysis time per sample.

The overall process discovering better enzymes can be summarized as the following steps. First, several mutations are made to the desired enzyme. Next, the enzymes are produced and reacted with the precursor chemical. Further, each of the individual reactions is measured for product production with a low-throughput technique. Finally, the most successful mutant becomes the starting enzyme for the first step.

Using error prone PCR, degenerate oligonucleotide incorporation, large scale DNA synthesis or other mutation generation strategies allows millions or billions of mutations to be rapidly made to the starting enzyme. However, because the low-throughput measurement techniques require that each enzyme be physically separated from the other enzyme variants it is impossible to evaluate each of these enzymes. At most thousands of enzyme variants can be evaluated in this way per day. While this rate of enzyme evaluation has produced enzymes of astounding value, the possibilities would be staggering if it were feasible to evaluate each of the million to billion enzyme variants generated.

Enzyme evaluation can take place within cells or in cell-free systems. The choice of cellular or cell-free enzyme optimization may depend on enzyme stability, cofactor and precursor availability and product or precursor toxicity, solubility, or transport. When large numbers of cofactors are needed or the enzyme is unstable outside of a cell then the optimization process will be carried out intracellularly. When the product compound is toxic or the enzymes are destined for extracellular use then optimization in a cell-free system is ideal.

When optimization is carried out within the cell, biosensor-based methods exist to enable high-throughput evaluation of enzyme mutants. These biosensors enable millions or billions of cells to be evaluated in a single day. Biosensors are genetically encoded sensors that turn on or off protein production proportional to the amount of product molecule they observe. When the protein they turn on is a fluorescent protein then rapid fluorescence measurement techniques can be used to evaluate a cell for its capacity to produce the product. When the protein the biosensor turns on is an antidote protein, toxin exposure can be used to find the most productive cells. However no analogous method exists for rapid evaluation of enzyme outside of a cell. There is a great need for a cell-free biosensor-based method that enables high-throughput evaluation of enzyme mutants for enzyme discovery and optimization.

### SUMMARY

The present disclosure addresses this need and is based on the discovery that a cell-free biosensor-based method can be used for high-throughput evaluation of enzyme mutants for enzyme discovery and optimization. The present disclosure provides a method of selecting a subset of enzyme variants for the production of a metabolite including providing a plurality of a first nucleotide sequence each encoding a different enzyme variant, providing a precursor molecule wherein the enzyme variants when expressed convert the precursor molecule to the metabolite, providing a second nucleotide sequence encoding a sensor biomolecule, providing a third nucleotide sequence encoding a reporter, wherein the sensor biomolecule when expressed interacts with the metabolite and induces the expression of the reporter in a manner dependent on the concentration of the produced metabolite, and screening the enzyme variants by detecting the reporter to identify a subset of enzyme variants. In one aspect, a method of selecting a candidate enzyme variant from a library of enzyme variants for the production of a metabolite is provided. In one embodiment, the method comprises providing a plurality of first nucleotide sequences each encoding a different enzyme variant of the library, providing a precursor molecule wherein the enzyme variant when expressed converts the precursor molecule to the metabolite, providing a second nucleotide sequence encoding a sensor biomolecule, providing a third nucleotide sequence encoding a reporter, wherein the sensor biomolecule when expressed interacts with the metabolite and induces the expression of the reporter in a manner dependent on the concentration of the produced metabolite, and screening the enzyme variants by detecting the reporter to identify the candidate enzyme variant.

The present disclosure provides a method wherein the enzyme variants convert the precursor molecule to the metabolite directly or through one or more intermediate steps. The present disclosure provides a method wherein one or more of the enzyme variants are completely or partially randomized. The present disclosure provides a method wherein the first, second or third nucleotide sequence is DNA or RNA. The present disclosure provides a method wherein the DNA and/or RNA is linear or included on a plasmid. The present disclosure provides a method wherein the nucleotide sequences can be physically separated or attached or any combination thereof. The present disclosure provides a method wherein cofactors are further provided. The present disclosure provides a method wherein the enzyme variants, the sensor biomolecule and the reporter are produced using a cell-free expression system. The present disclosure provides a method wherein the enzyme variants, the sensor biomolecule and the reporter can be produced directly in an evaluation vessel. The present disclosure provides a method wherein the evaluation vessel is in an emulsion or microtiter well format. The present disclosure provides a method wherein the enzyme variants, the sensor biomolecule and the reporter can be produced outside and then combined in an evaluation vessel. The present disclosure provides a method wherein the cell-free expression system comprising commercially available in vitro translation reagents and/or kits. The present disclosure provides a method wherein the subset of enzyme variants are validated by sequencing. The present disclosure provides a method wherein enzyme variants and/or sensor biomolecules are provided. The present disclosure provides a method wherein the selection process is repeated on the subset of identified enzyme variants for optimization. The present disclosure provides a method wherein the reporter is a fluorescent protein. The present disclosure provides a method wherein the fluorescent protein is GFP. The present disclosure provides a method wherein the reporter is a member selected from the group consisting of mPlum, mCherry, tdTomato, mStrawberry, J-Red, DsRed-monomer, mOrange, mKO, mCitrine, Venus, YPet, EYFP, Emerald, EGFP, CyPet, mCFPm, Cerulean, T-Sapphire, Firefly (FLuc), modified firefly (Ultra-Clo), Click beetle (CBLuc), Sea pansy (RLuc), Copepod crustacean (GLuc), and Ostracod crustacean (CLuc). The present disclosure provides a method wherein the reporter further comprises luciferase for detection by light, pigments for detection by color, surfactants for detection by emulsion breaking, and adhesives for detection by adhesion. The present disclosure provides a method wherein the screening is carried out by fluorescent microscopy, microtiter plate assay, emulsion assay, microfluidic assay, pull-down assay or luciferase high throughput screening. The present disclosure provides a method wherein the sensor biomolecule and the metabolite binding partner is a member pair selected from the group consisting of AcuR/acrylate, cdaR/glucaric acid, ttgR/naringennin, ttgR/phenol, btuB riboswitch/cobalamin, mphR/macrolides, tetR/tetracycline derivates, benM/muconic acid, alkS/medium chain n-alkanes, xylR/xylose, araC/Arabinose, gntR/Gluconate, galS/Galactose, trpR/tryptophan, qacR/Berberine, rmrR/Phytoalexin, cymR/Cumate, melR/Melibiose, rafR/Raffinose, nahR/Salicylate, nocR/Nopaline, clcR/Chlorobenzoate, varR/Virginiamycin, rhaR/Rhamnose, PhoR/Phosphate, MalK/Malate, GlnK/Glutamine, Retinoic acid receptor/Retinoic acid, Estrogen receptor/Estrogen and Ecdysone receptor/Ecdysone. The present disclosure provides a method wherein the sensor biomolecule is a transcription factor, riboswitch, two-component signaling protein, a nuclear hormone receptor, a G-protein coupled receptor, a periplasmic binding protein, or an engineered protein switch. The present disclosure provides a method wherein the sensor biomolecule is cdaR and the metabolite is a diacid. The present disclosure provides a method wherein the biosensor is an engineered protein switch such as an engineered calmodulin. The present disclosure provides a method wherein the sensor is AcuR and the metabolite is acrylate. The present disclosure provides a method wherein the enzyme is PCS, MIOX, Udh, or INO1. The present disclosure provides a method wherein the precursor molecule is 3-hydroxypropionate. The present disclosure provides a method wherein the reporter protein is an emulsion-breaking protein. The present disclosure provides a method wherein the plurality of the first nucleotide sequences encoding the different enzyme variants are generated by methods comprising gene synthesis, error prone PCR, targeted mutagenesis, or oligonucleotide directed mutagenesis.

The present disclosure further provides a method of identifying a subset of sensor biomolecule variants for a metabolite including providing a plurality of a first nucleotide sequence each encoding a different sensor biomolecule variants, providing a metabolite, providing a second nucleotide sequence encoding a reporter, wherein the sensor biomolecule variant when expressed interacts with the metabolite and induces the expression of the reporter in a manner dependent on the concentration of the produced metabolite, and screening the sensor biomolecule variants by detecting the reporter to identify a subset of sensor biomolecule variants. In one aspect, a method of identifying a candidate sensor biomolecule variant from a library of sensor biomolecule variants for a metabolite is provided. The method comprises providing a plurality of first nucleotide sequences each encoding a different sensor biomolecule variant of the library of sensor biomolecule variants, providing a metabolite, providing a second nucleotide sequence encoding a reporter, wherein the sensor biomolecule variant when expressed interacts with the metabolite and induces the expression of the reporter in a manner dependent on the concentration of the produced metabolite, and screening the sensor biomolecule variants by detecting the reporter to identify the candidate sensor biomolecule variant.

The present disclosure provides a cell-free bio-sensing system for selecting a subset of enzyme variants for the production of a metabolite including a plurality of a first nucleotide sequence each encoding a different enzyme variant, a precursor molecule wherein the enzyme variant when expressed converts the precursor molecule to the metabolite, a second nucleotide sequence encoding a sensor biomolecule, a third nucleotide sequence encoding a reporter,

wherein the sensor biomolecule when expressed interacts with the metabolite and induces the expression of the reporter in a manner dependent on the concentration of the produced metabolite, and wherein the enzyme variants are screened by detecting the reporter to identify a subset of enzyme variants. In one aspect, a cell-free bio-sensing system for selecting a candidate enzyme variant from a library of enzyme variants for the production of a metabolite is provided. In one embodiment, the cell-free bio-sensing system comprises a plurality of first nucleotide sequences each encoding a different enzyme variant of the library of enzyme variants, a precursor molecule wherein the enzyme variant when expressed converts the precursor molecule to the metabolite, a second nucleotide sequence encoding a sensor biomolecule, a third nucleotide sequence encoding a reporter, wherein the sensor biomolecule when expressed interacts with the metabolite and induces the expression of the reporter in a manner dependent on the concentration of the produced metabolite, and wherein the enzyme variants are screened by detecting the reporter to identify the candidate enzyme variant.

The present disclosure also provides a cell-free bio-sensing system for identifying a subset of sensor biomolecule variants for a metabolite including a plurality of a first nucleotide sequence each encoding a different sensor biomolecule variant, a metabolite, a second nucleotide sequence encoding a reporter, wherein the sensor biomolecule variant when expressed interacts with the metabolite and induces the expression of the reporter in a manner dependent on the concentration of the produced metabolite, and wherein the sensor biomolecule variants are screened by detecting the reporter to identify a subset of sensor biomolecule variants. The present disclosure provides a system wherein the enzyme variants convert the precursor molecule to the metabolite directly or through one or more intermediate steps. The present disclosure provides a system wherein one or more of the enzyme variants are completely or partially randomized. The present disclosure provides a system wherein the first, second or third nucleotide sequence is DNA or RNA. The present disclosure provides a system wherein the DNA and/or RNA is linear or included on a plasmid. The present disclosure provides a system wherein the nucleotide sequences can be physically separated or attached or any combination thereof. The present disclosure provides a system further comprises cofactors. The present disclosure provides a system wherein the enzyme variants, the sensor biomolecule and the reporter are produced using a cell-free expression system. The present disclosure provides a system wherein the enzyme variants, the sensor biomolecule and the reporter can be produced directly in an evaluation vessel. The present disclosure provides a system wherein the evaluation vessel is in an emulsion or microtiter well format. The present disclosure provides a system wherein the enzyme variants, the sensor biomolecule and the reporter can be produced outside and then combined in an evaluation vessel. The present disclosure provides a system wherein the cell-free expression system comprising commercially available in vitro translation reagents and/or kits. The present disclosure provides a system wherein the subset of enzyme variants are validated by sequencing. The present disclosure provides a system wherein enzyme variants and/or sensor biomolecules are provided. The present disclosure provides a system wherein the selection process is repeated on the subset of identified enzyme variants for optimization. The present disclosure provides a system wherein the reporter is a fluorescent protein. The present disclosure provides a system wherein the fluorescent protein is GFP. The present disclosure provides a system wherein the reporter is a member selected from the group consisting of mPlum, mCherry, tdTomato, mStrawberry, J-Red, DsRed-monomer, mOrange, mKO, mCitrine, Venus, YPet, EYFP, Emerald, EGFP, CyPet, mCFPm, Cerulean, T-Sapphire, Firefly (FLuc), modified firefly (Ultra-Clo), Click beetle (CBLuc). Sea pansy (RLuc), Copepod crustacean (GLuc), and Ostracod crustacean (CLuc). The present disclosure provides a system wherein the reporter further comprises luciferase for detection by light, pigments for detection by color, surfactants for detection by emulsion breaking, and adhesives for detection by adhesion. The present disclosure provides a system wherein the screening is carried out by fluorescent microscopy, microtiter plate assay, emulsion assay, microfluidic assay, pull-down assay or luciferase high throughput screening. The present disclosure provides a system wherein the sensor biomolecule and the metabolite binding partner is a member pair selected from the group consisting of AcuR/acrylate, cdaR/glucaric acid, ttgR/naringennin, ttgR/phenol, btuB riboswitch/cobalamin, mphR/macrolides, tetR/tetracycline derivates, benM/muconic acid, alkS/medium chain n-alkanes, xylR/xylose, araC/Arabinose, gntR/Gluconate, galS/Galactose, trpR/tryptophan, qacR/Berberine, rmrR/Phytoalexin, cymR/Cumate, melR/Melibiose, rafR/Raffinose, nahR/Salicylate, nocR/Nopaline, clcR/Chlorobenzoate, varR/Virginiamycin, rhaR/Rhamnose, PhoR/Phosphate, MalK/Malate, GlnK/Glutamine, Retinoic acid receptor/Retinoic acid, LacI/allolactose, Estrogen receptor/Estrogen and Ecdysone receptor/Ecdysone. The present disclosure provides a system wherein the sensor biomolecule is a transcription factor, riboswitch, two-component signaling protein, a nuclear hormone receptor, a G-protein coupled receptor, a periplasmic binding protein, or an engineered protein switch. The present disclosure provides a system wherein the sensor biomolecule is cdaR and the metabolite is a diacid. The present disclosure provides a system wherein the biosensor is an engineered protein switch such as an engineered calmodulin. The present disclosure provides a system wherein the sensor is AcuR and the metabolite is acrylate. The present disclosure provides a system wherein the enzyme is PCS, MIOX, Udh, or INO1. The present disclosure provides a system wherein the precursor molecule is 3-hydroxypropionate. The present disclosure provides a system wherein the reporter protein is an emulsion-breaking protein. The present disclosure provides a system wherein the plurality of the first nucleotide sequences encoding the different enzyme variants are generated by methods comprising gene synthesis, error prone PCR, targeted mutagenesis, or oligonucleotide directed mutagenesis.

In another aspect, a cell-free bio-sensing system for identifying a candidate sensor biomolecule variant from a library of sensor biomolecule variants for a metabolite is provided. In one embodiment, the cell-free bio-sensing system comprises a plurality of first nucleotide sequences each encoding a different sensor biomolecule variant of the library of sensor biomolecule variants, a metabolite, a second nucleotide sequence encoding a reporter, wherein the sensor biomolecule variant when expressed interacts with the metabolite and induces the expression of the reporter in a manner dependent on the concentration of the produced metabolite, and wherein the sensor biomolecule variants are screened by detecting the reporter to identify the candidate sensor biomolecule variant.

In some embodiments, the enzyme variants or the first nucleotide sequences encoding the enzyme variants are attached to a solid support for multiplex screening of candidate enzyme variants. In other embodiments, the solid support comprises multiple compartments in membrane, filter, paper, gel, plate, slide format and the like. In exemplary embodiments, an individual enzyme variant or an individual nucleotide sequence encoding the enzyme variant is trapped in an individual compartment of the multi-compartment solid support. In one embodiment, the enzyme variant is isolated with corresponding precursor molecules and reporter sequences inside an individual compartment. In some embodiments, each individual compartment is immobilized, or temporarily immobilized, within the multi-compartment solid support. In other embodiments, the individual compartment can be sorted by an automated sorting system. In certain embodiments, the individual compartment can be separated from the multi-compartment solid support by manual extraction. In other embodiments, the candidate enzyme variant can be identified based on the known content of each individual compartment, or by targeted sequencing, or in-situ imaging.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 shows fluorescence signal after addition of acrylate.

FIG. 2 shows the result of an in vitro reaction using the addition of purified protein sensor according to an embodiment of the disclosure.

### DETAILED DESCRIPTION

The present disclosure provides methods and systems that enable high-throughput enzyme evaluation and discovery to be carried out in cell-free systems. The present disclosure provides a method to make biosensors work without the need for living cells. The biosensors operate in in vitro cell-free systems producing a reporter protein, most commonly a fluorescent protein, in response to the presence of a product compound. The disclosure provides a method that enables enzyme evaluation rates from a thousand up to million enzymes per minute.

Because cells are not involved in the enzyme evaluation process several difficulties in using cell-based systems are obviated. First, problems regarding transport of the precursor molecules into a cell are avoided because there are no membranes in the cell-free system. Second, precursor and product molecule concentrations are not constrained to levels that would be non-toxic to a cell. Many products are highly toxic to cells at the desired production concentration and only by using a cell-free system are these high levels of production attainable. Third, the chemical environment in which the reaction is happening is completely defined allowing the chemical production process to approximate the field of chemistry more than biology.

The process to enable high-throughput cell-free enzyme evaluation can be summarized in the following major steps:

1. Enzyme variants are produced in vitro from DNA or RNA using a mixture of dNTPs, polymerases, translation machinery, cofactors and energy sources.

2. Product precursor and necessary cofactors are introduced to the mixture and incubated for an appropriate period of time.

3. Biosensor protein is added to the mixture with the corresponding reporter DNA.

4. The complete mixture is incubated while reporter is produced at a rate proportional to enzyme productivity.

5. The mixture is evaluated for reporter activity (generally the level fluorescence in the case where the reporter is a fluorescent protein).

At this point the best enzyme variant can be used to generate a series of mutants that can be evaluated in this manner again. The timing of Steps 1 through 3 can be varied to achieve different effects. In Step 3 the biosensor may be added as a purified or crude protein or as genetic material such as DNA or RNA that is then turned into protein by the protein producing system that is already present in the cell-free mixture. The DNA sequences encoding the enzymes, the biosensor and the reporter can be on linear or circular DNA, but must conform to specific design considerations. Cell-free protein expression reagents (polymerases, cofactors, energy sources, etc) can be purchased separately or from any of the commercially available kits.

The cell-free biosensors according to the present disclosure are used to rapidly identify better enzyme variants in micro-titer plates. Three standard 384-well micro-titer plates allow evaluation of more than 1,000 enzyme variants in less than a minute. Each well of the micro-titer plates contains a different enzyme mutant. The wells that contain enzyme mutants that produce higher amounts of product compound fluorescence more brightly. The micro-titer plate reader readily identifies these wells enabling the scientist to harvest these new and more powerful enzymes. Just one or multiple enzymes can be evolved simultaneously.

The cell-free biosensors according to the present disclosure are also deployed in conjunction with emulsion-based sorting technology to enable enzyme evaluation rates of more than a million enzymes per minute. A master mixture of cell-free translation components, the precursor molecule, reporter DNA and the biosensor is created. The DNA of the enzymes to be evaluated is added to a mixture at a very low concentration. This enzyme DNA is multiplexed such that the individual mutants are mixed together—multiplexing enables a several order magnitude increase in throughput when compared to singleplex analysis. The final evaluation mixture is mixed and allocated to droplet using standard emulsion protocols. Each droplet contains a single enzyme mutant because the concentration of the enzyme DNA is so low in the original mix. The emulsion is incubated for a short period of time resulting in a range of droplet fluorescence intensities. High fluorescence droplets indicate high biosensor activity in those individual droplets. This high biosensor activity is indicative of a high quality enzyme variant that is more productive than other enzyme variants. The droplets can be flowed through an apparatus that evaluates each droplet's fluorescence and retains only those droplets with predetermined level of fluorescent intensity. These devices are common in the literature. The case above relies on a fluorescent protein as the reporter protein. In the case where the reporter protein is an emulsion-breaking protein the highest quality enzyme variants will be able to break out of the emulsions and can be retained without the need for any droplet sorting.

The cell-free biosensing technique according to the present disclosure also enables the rapid discovery of new biosensors. Biosensor mutants are evaluated for new substrate specificity and response behavior when the variable component of the evaluation mixture is the biosensor itself. In these cases the product molecule is supplied rather than the precursor molecule.

The term “biosensor”, as used herein, generally refers to genetically encoded devices that monitor the intracellular concentration of a specific compound. Biosensors produce fluorescence or another readout proportional to the concentration of that compound within the cell.

The term “multiplex”, as used herein, generally refers to a process in biology that operates on many distinct elements (e.g., cells, DNA molecules or metabolites) that coexist in space and time. Multiplexing enables a single process to work on millions of elements with the same effort that would be required to carry out the process on a single element.

### Sensor/Metabolite Pairs

According to certain aspects, known sensor/metabolite pairs can be used in the fluorescent monitoring methods described herein where the binding of the sensor to the metabolite results in production of fluorescent molecules by the cell-free system. Exemplary known sensor/metabolite pairs include those shown in Table 1 below. Others are known in the art.

TABLE 1

| Sensor Gene | Molecule | Type of Sensor |
|---|---|---|
| cdaR | glucaric acid | Transcriptional activator |
| ttgR | naringennin | Transcriptional repressor |
| | (flavanoids) | |
| btuB riboswitch | cobalamin | Riboswitch |
| mphR | macrolides | Transcriptional repressor |
| tetR | tetracycline | Transcriptional repressor |
| | derivates | |
| benM | muconic acid | Transcriptional activator |
| alkS | medium chain | Transcriptional activator |
| | n-alkanes | |
| xylR | xylose | Transcriptional activator |
| araC | Arabinose | Transcriptional activator |
| gntR | Gluconate | Transcriptional repressor |
| galS | Galactose | Transcriptional repressor |
| trpR | tryptophan | Transcriptional repressor |
| qacR | Berberine | Transcriptional repressor |
| rmrR | Phytoalexin | Transcriptional repressor |
| cymR | Cumate | Transcriptional repressor |
| melR | Melibiose | Transcriptional activator |
| rafR | Raffinose | Transcriptional activator |
| nahR | Salicylate | Transcriptional activator |
| nocR | Nopaline | Transcriptional activator |
| clcR | Chlorobenzoate | Transcriptional activator |
| varR | Virginiamycin | Transcriptional repressor |
| rhaR | Rhamnose | Transcriptional repressor |
| PhoR | Phosphate | Two-component system |
| MalK | Malate | Two-component system |
| GlnK | Glutamine | Two-component system |
| Retinoic acid receptor | Retinoic acid | Nuclear hormone receptor |
| Estrogen receptor | Estrogen | Nuclear hormone receptor |
| Ecdysone receptor | Ecdysone | Nuclear hormone receptor |

According to certain aspects described herein, sensor/metabolite pairs can be selected based upon the following considerations: (1) the relationship between stimulus strength and circuit activation; (2) the response time of the biosensor to a stimulus; (3) the heterogeneity of biosensor activation between cells in an isogenic population and/or (4) the cross-reactivity with stimuli of other biosensors. Exemplary biosensors are useful DNA binding proteins having a cognate promoter/operator and that are induced by a target compound such as a metabolite that can be produced enzymatically through metabolic engineering.

It is to be understood that the examples of sensors and their corresponding metabolite binding partners are exemplary only and that one of skill in the art can readily identify additional sensors and their corresponding metabolite binding partners for use in the present disclosure. The transformed microorganism is intended to express the sensors and the metabolite under suitable conditions.

The biosynthetic pathways for production of any particular metabolite binding partner are known to those of skill in the art. The sensor sequence is known to those of skill in the art, such as being based on a published literature search. For example, nucleic acid and amino acid sequences for the above metabolite binding partners/sensors, or the nucleic acid and amino acid sequences for biosynthetic pathways that produce certain metabolites are fully described in the following: cdaR (Monterrubio et al. 2000 J. Bacteriol 182(9):2672-4), tetR (Lutz and Bujard Nucleic Acids Res. 1997 25(6):1203-10), alkS (Canosa et al. Mol Micriobiol 2000 35(4):791-9), ttgR (Teran, et al. Antimicrob Agents Chemother. 47(10):3067-72 (2003)), btuB riboswitch (Nahvi, et al. Nucleic Acids Res. 32:143-150 (2004)); glucaric acid (Moon, et al. Appl Env Microbiol. 75:589-595 (2009)), naringenin (Santos, et al. Metabolic Engineering. 13:392-400 (2011)), alkanes (Steen, et al. 463:559-562 (2009)), cobalamin (Raux, et al. Cell Mol Life Sci. 57:1880-1893. (2000)), muconic acid (Niu, et al. Biotechnol Prog. 18:201-211. (2002)) each of which are hereby incorporated by reference in its entirety. Methods described herein can be used to insert the nucleic acids into the genome of the microorganism that are responsible for production of sensors, metabolite binding partners and biosynthetic pathways.

Standard recombinant DNA and molecular cloning techniques used herein are well known in the art and are described in Sambrook, J., Fritsch, E. F. and Maniatis, T., Molecular Cloning: A Laboratory Manual, 2^{nd} ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety.

Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

Microorganisms may be genetically modified to delete genes or incorporate genes by methods known to those of skill in the art. Vectors and plasmids useful for transformation of a variety of host cells are common and commercially available from companies such as Invitrogen Corp. (Carlsbad, Calif.), Stratagene (La Jolla, Calif.), New England Biolabs, Inc. (Beverly, Mass.) and Addgene (Cambridge, Mass.).

Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IP_{L}, IP_{R}, T7, tac, and trc (useful for expression in Escherichia coli and Pseudomonas); the amy, apr, npr promoters and various phage promoters useful for expression in Bacillus subtilis, and Bacillus licheniformis; nisA (useful for expression in Gram-positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)). Termination control regions may also be derived from various genes native to the preferred hosts.

Certain vectors are capable of replicating in a broad range of host bacteria and can be transferred by conjugation. The complete and annotated sequence of pRK404 and three related vectors-pRK437, pRK442, and pRK442(H) are available. These derivatives have proven to be valuable tools for genetic manipulation in Gram-negative bacteria (Scott et al., Plasmid 50(1):74-79 (2003)). Several plasmid derivatives of broad-host-range Inc P4 plasmid RSF1010 are also available with promoters that can function in a range of Gram-negative bacteria. Plasmid pAYC36 and pAYC37, have active promoters along with multiple cloning sites to allow for the heterologous gene expression in Gram-negative bacteria.

Chromosomal gene replacement tools are also widely available. For example, a thermosensitive variant of the broad-host-range replicon pWV101 has been modified to construct a plasmid pVE6002 which can be used to create gene replacement in a range of Gram-positive bacteria (Maguin et al., J. Bacteriol. 174(17):5633-5638 (1992)). Additionally, in vitro transposomes are available to create random mutations in a variety of genomes from commercial sources such as EPICENTRE® (Madison, Wis.).

Vectors useful for the transformation of E. coli are common and commercially available. For example, the desired genes may be isolated from various sources, cloned onto a modified pUC19 vector and transformed into E. coli host cells. Alternatively, the genes encoding a desired biosynthetic pathway may be divided into multiple operons, cloned onto expression vectors, and transformed into various E. coli strains.

The Lactobacillus genus belongs to the Lactobacillales family and many plasmids and vectors used in the transformation of Bacillus subtilis and Streptococcus may be used for Lactobacillus. Non-limiting examples of suitable vectors include pAM.beta.1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol. 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (van Kranenburg R, Golic N, Bongers R, Leer R J, de Vos W M, Siezen R J, Kleerebezem M. Appl. Environ. Microbiol. 2005 March; 71(3): 1223-1230), which may be used for transformation.

Initiation control regions or promoters, which are useful to drive expression of the relevant pathway coding regions in the desired Lactobacillus host cell, may be obtained from Lactobacillus or other lactic acid bacteria, or other Gram-positive organisms. A non-limiting example is the nisA promoter from Lactococcus. Termination control regions may also be derived from various genes native to the preferred hosts or related bacteria.

The various genes for a desired biosynthetic or other desired pathway may be assembled into any suitable vector or vectors, such as those described above. A single vector need not include all of the genetic material encoding a complete pathway. One or more or a plurality of vectors may be used in any aspect of genetically modifying a cell as described herein. The codons can be optimized for expression based on the codon index deduced from the genome sequences of the host strain, such as for Lactobacillus plantarum or Lactobacillus arizonensis. The plasmids may be introduced into the host cell using methods known in the art, such as electroporation, as described in any one of the following references: Cruz-Rodz et al. (Molecular Genetics and Genomics 224:1252-154 (1990)), Bringel and Hubert (Appl. Microbiol. Biotechnol. 33: 664-670 (1990)), and Teresa Alegre, Rodriguez and Mesas (FEMS Microbiology Letters 241:73-77 (2004)). Plasmids can also be introduced to Lactobacillus plantatrum by conjugation (Shrago, Chassy and Dobrogosz Appl. Environ. Micro. 52: 574-576 (1986)). The desired biosynthetic pathway genes can also be integrated into the chromosome of Lactobacillus using integration vectors (Hols et al. Appl. Environ. Micro. 60:1401-1403 (1990); Jang et al. Micro. Lett. 24:191-195 (2003)).

Microorganisms which may serve as host cells and which may be genetically modified to produce recombinant microorganisms as described herein may include one or members of the genera Clostridium, Escherichia, Rhodococcus, Pseudomonas, Bacillus, Lactobacillus Saccharomyces, and Enterococcus. Particularly suitable microorganisms include Escherichia coli, Bacillus subtilis, and Saccharomyces cerevisiae.

### Methods for Screening Using a Reporter

Methods described herein utilize the detection and measurement of detectable reporters. Exemplary detectable reporters include fluorescent molecules or fluorescent proteins. Exemplary fluorescent reporters include those identified in Shaner et al., Nature methods, Vol. 2, No. 12, pp. 905-909 (2005) hereby incorporated by reference in its entirety. An exemplary list of fluorescent reporters known to those of skill in the art includes mPlum, mCherry, tdTomato, mStrawberry, J-Red, DsRed-monomer, mOrange, mKO, mCitrine, Venus, YPet, EYFP, Emerald, EGFP, CyPet, mCFPm, Cerulean and T-Sapphire, and the like.

Exemplary non-fluorescent, but light emitting reporters include luciferase and its derivatives such as those disclosed in Thorne et al., Chemistry and Biology, vol. 17, issue 6, pp. 646-657 (2010) hereby incorporated by reference in its entirety. An exemplary list of non-fluorescent reporter known to those of skill in the art include Firefly (FLuc), modified firefly (Ultra-Clo), Click beetle (CBLuc), Sea pansy (RLuc), Copepod crustacean (GLuc), Ostracod crustacean (CLuc) and the like.

The disclosure provides biosensors that link metabolite levels to fluorescent protein expression and enable fluorescence-based screens. The biosensor-based screens according to the present disclosure can provide evaluation rates of up to 1×10⁹ designs per day. Fluorescent screening can be evaluated with fluorescent plate readers in 96 or 384-well plates and is useful for prototyping the screening system. These cell-free fluorescent screening can be used for the next round of design or chosen for a commercial production system.

Further methods include microtiter plate assays, for example where screening by fluorescence is done robotically in microtiter plates, such as 1536-well plates or 9600 well plates. Such methods may be combined with robotic handling and advanced plate readers typical of high throughput screening. Further methods include emulsion assays, for example, where the reaction can be trapped within emulsions and assayed using microfluidics, such as described in Wang et al., Nature Biotechnology, Volume 32, pp. 473-478 (2014) hereby incorporated by reference in its entirety. Other microfluidic assays can be used to evaluate screening such as those described in Guo et al., Lab Chip, 2012, 12, 2146-2155 hereby incorporated by reference in its entirety.

Other methods and assays which do not rely on fluorescent or light emitting reporters can be used to detect reaction rates according to the methods described herein. Such methods include those that use transcription but not necessarily fluorescence or luminescence. Exemplary methods include pull-down assays that can measure high metabolite production capabilities. Further methods include luciferase high-throughput screening such as described in Fan et al., ASSAY and Drug Development Technologies, Volume 5, Number 1, pp. 127-136 (2007) hereby incorporated by reference in its entirety.

The above disclosure generally describes the present invention. All references disclosed herein are expressly incorporated by reference. The following examples are set forth as being representative of the present disclosure. These examples are not to be construed as limiting the scope of the present disclosure as these and other equivalent embodiments will be apparent in view of the present disclosure, figures and accompanying claims.

### Solid Support

In certain embodiments, the enzyme variants or the nucleotide encoding the enzyme variants, or the sensor biomolecule variants and the like described herein can be immobilized on a support. The support can be simple square grids, checkerboard (e.g., offset) grids, hexagonal arrays and the like. Suitable supports include, but are not limited to, membranes, papers, filters, slides, beads, chips, particles, strands, gels, sheets, tubing, spheres, containers, capillaries, pads, slices, films, culture dishes, plates (e.g., 96-well, 48-well, 24-well, 12-well, eight-well, six-well, four-well, single-well and the like), and the like. In various embodiments, a solid support may be biological, nonbiological, organic, inorganic, or any combination thereof. The solid support can be made up of multiple individual compartments. In some embodiments, each of the individual compartment of the multi-compartment solid support can be immobilized or temporarily immobilized to the multi-compartment solid support. The immobilization facilitates sorting of the individual compartments. The immobilized individual compartment can be extracted or removed from the solid support, such as by manual extraction.

In certain embodiments, a support may have functional groups attached to its surface which can be used to bind one or more reagents described herein. One or more reagents can be attached to a support by hybridization, covalent attachment, magnetic attachment, affinity attachment and the like. Supports may also be functionalized using, for example, solid-phase chemistries known in the art (see, e.g., U.S. Pat. No. 5,919,523).

As used herein, the term “attach” refers to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994.

### Example I

### Cell-Free Observation of Acrylate

DNA encoded biological acrylate sensor was used to detect acrylate. AcuR is a transcription factor whose activity is dependent on the concentration of acrylate. A linear dsDNA encoding the acrylate sensor AcuR is combined with a reporter circular plasmid which contained a GFP protein controlled by a promoter regulated by AcuR. Thus, GFP fluorescence is related to the amount of acrylate in the sample.

In order to obtain an adequate signal-to-noise ratio (SNR), a T7 promoter was used to drive the expression of the sensor to achieve high expression of the sensor protein and ensure outnumbering of the number of reporter promoters, and well controlled expression. Additionally, an E. coli RNAP promoter was used to drive expression on the reporter, as it reduces the level of background signal, and provides with a more accurate control by AcuR.

### Methods

Linear DNA amplification: Linear dsDNA containing AcuR was prepared by PCR amplification using primers which contain a T7 promoter, and ribosome binding site (RBS).

Cell free preparation: In ice, a cell-free reaction was first prepared containing 10 uL of component A (NEB, P/N E6800), 7.5 uL of component B (NEB, P/N E6800), 5 U of murine RNAse inhibitor (NEB, P/N M0314S), and 2.5 U of E. coli RNA polymerase holoenzyme (NEB, P/N M0551S). A combined 250 ng of linear dsDNA encoding AcuR, 250 ng of circular plasmid containing the reporter GFP was added into the cell-free system reaction. Sequences of the DNA elements are attached below.

Finally, the sample was separated into two different aliquots, and different amounts of sodium acrylate solution was added to achieve concentrations of 0 mM, and 0.1 mM, respectively.

The samples were incubated at 37° C., and the fluorescence signal (395 nm excitation, 470 nm absorption) of the sample was measured every two minutes for 4 hours.

### Results

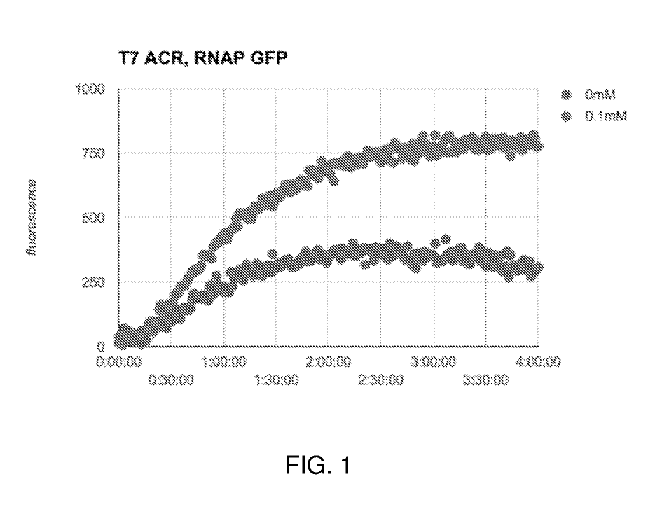

An increase of fluorescence to 750 arbitrary units in the sample containing acrylate was observed, and an increase of fluorescence to 260 arbitrary units in the sample that does not contain acrylate was observed (FIG. 1).

Reporter promoter:
(SEQ ID NO: 1)
GCTTCACAACCGCACTTGATTTAATAGACCATACCGTCTATTATTTCTGG
Reporter RBS:
(SEQ ID NO: 2)
Tttaactttaagaaggagatatacat
Reporter gene:
(SEQ ID NO: 3)
Atgcgtaaaggtgaagaactgttcaccggtgttgttccgatcctggttgaactggacggtgacgttaacggtcacaaattctctgttcgt
ggtgaaggtgaaggtgacgctaccaacggtaaactgaccctgaaattcatctgcaccaccggtaaactgccggttccgtggccgacc
ctggttaccaccctgacctacggtgttcagtgcttcgctcgttacccggaccacatgaaacagcacgacttcttcaaatctgctatgccg
gaaggttacgttcaggaacgtaccatctctttcaaagacgacggtacctacaaaacccgtgctgaagttaaattcgaaggtgacaccct
ggttaaccgtatcgaactgaaaggtatcgacttcaaagaagacggtaacatcctgggtcacaaactggaatacaacttcaactctcaca
acgtttacatcaccgctgacaaacagaaaaacggtatcaaagctaacttcaaaatccgtcacaacgttgaagacggttctgttcagctgg
ctgaccactaccagcagaacaccccgatcggtgacggtccggttctgctgccggacaaccactacctgtctacccagtctgttctgtct
aaagacccgaacgaaaaacgtgaccacatggttctgctggaattcgttaccgctgctggtatcacccacggtatggacgaactgtaca
aa
Promoter and sequence sensor:
Promoter Sequence
(SEQ ID NO: 4)
Aattaatacgactcactatagggagaccacaac
Protein Coding Sequence
(SEQ ID NO: 5)
ATGCCGCTGACCGACACCCCGCCGTCTGTTCCGCAGAAACCGCGTCGTGGTCGTC
CGCGTGGTGCTCCGGACGCTTCTCTGGCTCACCAGTCTCTGATCCGTGCTGGTCT
GGAACACCTGACCGAAAAAGGTTACTCTTCTGTTGGTGTTGACGAAATCCTGAAA
GCTGCTCGTGTTCCGAAAGGTTCTTTCTACCACTACTTCCGTAACAAAGCTGACTT
CGGTCTGGCTCTGATCGAAGCTTACGACACCTACTTCGCTCGTCTCCTCGACCAG
GCGTTCCTGGACGGTTCGCTGGCTCCGCTGGCTCGTCTGCGTCTGTTCACCCGTAT
GGCTGAAGAAGGTATGGCTCGTCACGGTTTCCGTCGTGGTTGCCTGGTTGGTAAC
CTGGGTCAGGAAATGGGTGCTCTGCCGGACGACTTCCGTGCTGCTCTGATCGGTG
TTCTGGAAACCTGGCAGCGTCGTACCGCTCAGCTGTTCCGTGAAGCTCAGGCTTG
CGGTGAACTGTCTGCTGACCACGACCCGGACGCTCTGGCTGAAGCTTTCTGGATC
GGTTGGGAAGGTGCTATCCTGCGTGCTAAACTGGAACTGCGTCCGGACCCGCTGC
ACTCTTTCACCCGTACCTTCGGTCGTCACTTCGTTACCCGTACCCAGGAATAA
Terminator Sequence
(SEQ ID NO: 6)
AAC CCC TTG GGG CCT CTA AAC GGG TCT TGA GGG GTT TTT TG
Primers used for AcuR amplification:
(SEQ ID NO: 7)
P1: aattaatacgactcactatagggagaccacaacATG CCG CTG ACC GAC ACC C
(SEQ ID NO: 8)
P2: CAA AAA ACC CCT CAA GAC CCG TTT AGA GGC CCC AAG GGG TTT TAT
TCC TGG GTA CGG GTA ACG

### Example II

### Real-Time Monitoring of Acrylate Production in a Cell-Free System

DNA encoded biological acrylate sensor was used to detect acrylate which has been synthesized in real time. A dsDNA linear template encoding the enzyme PCS downstream of a T7 promoter, and the precursor molecule 3-hydroxypropionate was added to the sensor system described in Example 1.

### Methods

Linear DNA amplification: Linear dsDNA containing AcuR and PCS was prepared by PCR amplification using primers which contain a T7 promoter, and ribosome binding site (RBS).

Cell free preparation: In ice, a cell-free reaction was first prepared containing 10 μL of component A (NEB, P/N E6800), 7.5 μL of component B (NEB, P/N E6800), 5 U of murine RNAse inhibitor (NEB, P/N M0314S), and 2.5 U of E. coli RNA polymerase holoenzyme (NEB, P/N M0551S). A combined 250 ng of linear dsDNA encoding AcuR, 250 ng of linear dsDNA encoding PCS, and 250 ng of circular plasmid containing the reporter GFP was added into the cell-free system reaction. Sequences of the DNA elements are attached below.

Finally, the sample was separated into two different aliquots, and different amounts of 3-hydroxypropionate were added to a solution to achieve concentrations of 0 mM, and 0.1 mM, respectively. The samples were incubated at 37° C., and the fluorescence signal (395 nm excitation, 470 nm absorption) of the sample was measured every two minutes for 4 hours.

Promoter and sequence PCS:
Promoter Sequence
(SEQ ID NO: 4)
Aattaatacgactcactatagggagaccacaac
Protein Coding Sequence
(SEQ ID NO: 9)
ATGATCGATACCGCACCGCTGGCACCGCCGCGTGCTCCGCGCAGCAATCC
GATTCGTGATCGCGTGGATTGGGAAGCGCAGCGTGCAGCAGCACTGGCCG
ATCCGGGTGCATTTCATGGTGCGATCGCCCGTACCGTTATTCACTGGTAT
GATCCGCAGCATCACTGCTGGATTCGCTTCAACGAAAGCTCTCAGCGTTG
GGAAGGTCTGGATGCAGCAACGGGTGCTCCGGTTACAGTGGATTATCCTG
CCGATTACCAGCCGTGGCAGCAGGCATTTGATGATAGTGAAGCGCCGTTT
TATCGCTGGTTCAGCGGCGGTCTGACGAACGCATGTTTTAATGAAGTTGA
TCGTCACGTGACAATGGGTTACGGCGATGAAGTGGCGTATTACTTCGAAG
GTGATCGCTGGGATAATAGCCTGAACAATGGCCGTGGCGGTCCGGTGGTT
CAGGAAACGATTACCCGTCGCCGTCTGCTGGTTGAAGTGGTTAAAGCAGC
GCAGGTTCTGCGCGATCTGGGCCTGAAAAAAGGTGATCGTATCGCGCTGA
ACATGCCGAATATCATGCCGCAGATTTATTACACCGAAGCCGCAAAACGC
CTGGGTATTCTGTATACGCCGGTGTTTGGCGGTTTCAGTGATAAAACCCT
GAGCGATCGCATCCATAATGCAGGTGCGCGTGTGGTTATTACCTCTGATG
GCGCGTATCGTAACGCCCAGGTGGTTCCGTATAAAGAAGCCTACACGGAT
CAGGCACTGGATAAATACATCCCGGTGGAAACCGCCCAGGCAATTGTTGC
ACAGACGCTGGCAACCCTGCCGCTGACCGAAAGTCAGCGCCAGACGATTA
TCACCGAAGTGGAAGCAGCACTGGCAGGTGAAATTACGGTTGAACGTTCT
GATGTTATGCGCGGTGTGGGCAGTGCGCTGGCCAAACTGCGCGATCTGGA
TGCCAGTGTGCAGGCAAAAGTTCGTACCGTGCTGGCACAGGCGCTGGTTG
AAAGCCCGCCGCGCGTGGAAGCAGTGGTTGTGGTTCGTCATACGGGTCAG
GAAATCCTGTGGAATGAAGGCCGTGATCGCTGGAGCCACGATCTGCTGGA
TGCAGCACTGGCGAAAATTCTGGCTAACGCACGCGCCGCAGGTTTTGATG
TTCACTCTGAAAACGATCTGCTGAATCTGCCGGATGATCAGCTGATCCGT
GCTCTGTATGCGAGTATTCCGTGCGAACCAGTTGATGCCGAATATCCGAT
GTTTATTATCTACACGAGCGGTTCTACCGGCAAACCGAAAGGTGTTATTC
ATGTTCACGGCGGTTACGTGGCGGGCGTGGTTCATACCCTGCGCGTTAGT
TTCGATGCCGAACCGGGCGATACGATTTATGTGATCGCAGATCCGGGCTG
GATCACAGGTCAGAGCTACATGCTGACGGCAACCATGGCAGGTCGTCTGA
CTGGTGTGATTGCCGAAGGTTCTCCGCTGTTTCCGAGTGCGGGCCGCTAT
GCCTCTATTATCGAACGTTACGGTGTTCAGATTTTTAAAGCGGGCGTTAC
GTTCCTGAAAACCGTGATGAGTAACCCGCAGAATGTTGAAGATGTGCGCC
TGTATGATATGCACAGTCTGCGTGTGGCAACCTTTTGTGCAGAGCCGGTT
AGCCCGGCAGTGCAGCAGTTCGGTATGCAGATCATGACGCCGCAGTATAT
TAATAGCTACTGGGCGACGGAACATGGCGGTATTGTGTGGACCCACTTTT
ATGGCAACCAGGATTTCCCGCTGCGTCCAGATGCACATACGTACCCGCTG
CCGTGGGTTATGGGTGATGTTTGGGTGGCAGAAACCGATGAATCTGGCAC
CACGCGCTATCGCGTGGCGGATTTCGATGAAAAAGGTGAAATCGTTATCA
CCGCACCGTATCCGTACCTGACGCGAACCCTGTGGGGTGATGTGCCGGGT
TTTGAAGCGTATCTGCGTGGTGAAATCCCGCTGCGTGCATGGAAAGGTGA
TGCAGAACGTTTCGTTAAAACCTACTGGCGTCGTGGTCCGAATGGCGAAT
GGGGTTATATCCAGGGCGATTTTGCGATTAAATACCCGGATGGTAGTTTC
ACGCTGCATGGCCGCAGCGATGATGTTATTAATGTGTCCGGCCACCGTAT
GGGTACGGAAGAAATCGAAGGTGCCATTCTGCGTGATCGCCAGATCACCC
CGGATTCTCCGGTGGGTAACTGCATTGTGGTTGGCGCGCCGCATCGTGAA
AAAGGCCTGACCCCGGTTGCATTTATCCAGCCAGCACCGGGTCGTCACCT
GACGGGTGCAGATCGCCGTCGCCTGGATGAACTGGTGCGTACCGAAAAAG
GTGCAGTTAGCGTGCCGGAAGATTATATTGAAGTTAGTGCGTTTCCGGAA
ACCCGCAGCGGTAAATACATGCGTCGCTTCCTGCGTAATATGATGCTGGA
TGAACCGCTGGGCGATACCACGACCCTGCGCAACCCGGAAGTGCTGGAAG
AAATCGCGGCCAAAATTGCCGAATGGAAACGTCGCCAGCGCATGGCAGAA
GAACAGCAGATTATCGAACGTTATCGCTACTTTCGTATTGAATATCATCC
GCCGACCGCAAGTGCAGGTAAACTGGCAGTGGTTACGGTTACCAATCCGC
CGGTGAACGCCCTGAATGAACGTGCTCTGGATGAACTGAACACCATCGTG
GATCACCTGGCGCGTCGCCAGGATGTTGCAGCGATTGTGTTTACGGGTCA
GGGTGCTCGCAGCTTCGTGGCCGGTGCGGATATCCGTCAGCTGCTGGAAG
AAATTCATACCGTTGAAGAAGCCATGGCACTGCCGAACAATGCGCACCTG
GCCTTTCGCAAAATTGAACGTATGAACAAACCGTGCATTGCCGCAATCAA
TGGTGTGGCACTGGGCGGTGGCCTGGAATTTGCGATGGCCTGTCATTATC
GCGTTGCCGATGTGTACGCAGAATTTGGTCAGCCGGAAATCAACCTGCGT
CTGCTGCCGGGTTATGGTGGTACGCAGCGTCTGCCGCGTCTGCTGTACAA
ACGCAACAATGGTACAGGCCTGCTGCGTGCGCTGGAAATGATTCTGGGTG
GCCGCAGCGTGCCAGCAGATGAAGCACTGGAACTGGGTCTGATTGATGCA
ATCGCGACCGGCGATCAGGATAGTCTGAGCCTGGCCTGCGCACTGGCGCG
TGCGGCAATCGGTGCAGATGGTCAGCTGATTGAAAGCGCAGCGGTGACCC
AGGCCTTTCGTCATCGCCACGAACAGCTGGATGAATGGCGTAAACCGGAC
CCGCGCTTCGCGGATGATGAACTGCGCTCTATTATCGCCCATCCGCGTAT
CGAACGCATTATCCGTCAGGCGCATACCGTTGGTCGTGATGCAGCAGTGC
ACCGTGCACTGGATGCAATTCGTTATGGCATTATCCATGGTTTTGAAGCC
GGCCTGGAACACGAAGCAAAACTGTTCGCCGAAGCAGTGGTTGATCCGAA
TGGTGGCAAACGCGGCATCCGTGAATTTCTGGATCGTCAGTCTGCACCGC
TGCCGACACGTCGCCCGCTGATTACCCCGGAACAGGAACAGCTGCTGCGT
GATCAGAAAGAACTGCTGCCGGTGGGTAGTCCGTTTTTCCCTGGCGTTGA
TCGCATCCCGAAATGGCAGTATGCGCAGGCCGTGATTCGTGATCCCGATA
CTGGTGCAGCAGCACATGGCGATCCGATCGTTGCGGAAAAACAGATTATC
GTTCCGGTGGAACGTCCGCGTGCGAACCAGGCACTGATTTACGTTCTGGC
GAGCGAAGTGAACTTTAATGATATTTGGGCCATCACAGGTATTCCGGTGA
GCCGCTTCGATGAACATGATCGTGATTGGCACGTGACGGGTTCTGGTGGC
ATCGGCCTGATTGTTGCGCTGGGCGAAGAAGCCCGTCGCGAAGGTCGTCT
GAAAGTTGGCGATCTGGTGGCGATCTATAGCGGCCAGTCTGATCTGCTGA
GCCCGCTGATGGGTCTGGACCCGATGGCAGCCGATTTTGTGATTCAGGGT
AATGATACCCCGGATGGCTCTCATCAGCAGTTCATGCTGGCACAGGCACC
GCAGTGCCTGCCGATCCCGACGGATATGAGCATTGAAGCAGCGGGTTCTT
ATATCCTGAACCTGGGCACCATTTACCGCGCACTGTTTACGACCCTGCAA
Terminator Sequence
(SEQ ID NO: 6)
AAC CCC TTG GGG CCT CTA AAC GGG TCT TGA GGG GTT
TTT TG

### Example III

### Cell-Free Discovery of Enhanced Acrylate-Producing Enzymes

DNA encoded biological acrylate sensor was used to detect acrylate potential PCS mutants with higher activity of acrylate synthesis. A library of PCS enzymes was generated and the system described in Example 2 was used to detect mutants with higher production using the AcuR sensor activated GFP signal.

### Methods

Linear DNA amplification of AcuR: Linear dsDNA containing AcuR was prepared by PCR amplification using primers which contain a T7 promoter, and ribosome binding site (RBS).

PCS library generation: We generated a library of PCS enzymes combining error prone PCR, and degenerate primers on the catalytic center. We topo cloned the library, and purified 384 individual clones.

Cell free preparation: In ice, 384 cell-free reactions were prepared in a 384-well plate with V shaped bottom. 10 μL of component A (NEB, P/N E6800), 7.5 μL of component B (NEB, P/N E6800), 5 U of murine RNAse inhibitor (NEB, P/N M0314S), 2.5 U of E. coli RNA polymerase holoenzyme (NEB, P/N M0551S) were mixed with 3-hydroxypropionate to a concentration of 0.1 mM. A combined 250 ng of linear dsDNA encoding AcuR, 250 ng of plasmid encoding one PCS mutant, and 250 ng of circular plasmid containing the reporter GFP was added into the cell-free system reaction.

Finally, 3-hydroxypropionate was added to a solution to achieve concentrations of 0.1 mM. The samples were incubated at 37° C., and the fluorescence signal (395 nm excitation, 470 nm absorption) of the sample was measured every two minutes for 4 hours to each well. Wells showing higher fluorescence were analyzed.

Mutant identification: Wells with higher fluorescence are analyzed by PCR followed by sequence validation.

### Example IV

Cell-Free Discovery of Enhanced Acrylate-Producing Enzymes with Emulsion Sorting Technology

A large scale DNA encoded biological acrylate sensor was used to detect acrylate potential PCS mutants with higher activity of acrylate synthesis. A library of PCS enzymes was generated and the system described in example 2 was used to detect mutants with higher production using the AcuR sensor activated GFP signal.

### Methods

Linear DNA amplification of AcuR: Linear dsDNA containing AcuR was prepared by PCR amplification using primers which contain a T7 promoter, and ribosome binding site (RBS).

PCS library generation: A a library of PCS enzymes was generated combining error prone PCR, and degenerate primers on the catalytic center. Forward primer of PCS contains a 5′ biotin.

PCS library was emulsified in mineral oil with presence of beads. Emulsion PCR with biotin primers was performed to amplify the individual members of the library in each of the beads. The beads with successful amplification were recovered (ie https://www3.appliedbiosystems.com/cms/groups/mcb_support/documents/generaldocuments/cms_081748.pdf).

Cell free preparation: In ice, 384 cell-free reactions were prepared in a 384-well plate with V shaped bottom. 10 μL of component A (NEB, P/N E6800), 7.5 μL of component B (NEB, P/N E6800), 5 U of murine RNAse inhibitor (NEB, P/N M0314S), 2.5 U of E. coli RNA polymerase holoenzyme (NEB, P/N M0551S) were mixed with 3-hydroxypropionate to a concentration of 0.1 mM. A combined 250 ng of linear dsDNA encoding AcuR, 250 ng of circular plasmid containing the reporter GFP were added into the cell-free system reaction, and 3-hydroxypropionate was added to a solution to achieve concentrations of 0.1 mM. This mixture was emulsified with mineral oil, and the beads containing the PCS mutants.

The emulsion was incubated at 37° C. for 4 hours.

Mutant identification: Droplet sorter was used to separate droplets with higher fluorescence. Phenol chloroform extraction was used to separate aqueous phase from mineral oil, and PCR followed by sequence was used to identify the PCS mutants with higher activity.

TABLE 2
A list of natural and engineered biosensors by molecule sensed is provided.
Abbreviated sensor type names refer to the following: allosteric TF, allosteric
transcription factor; two-component, two-component systems; FRET, fluorescence
resonance energy transfer; GPCR, G-protein coupled receptor.

| | | | | |
|---|---|---|---|---|
| Molecule(s) | Molecule type | Sensor | Sensor type | Reference |

Natural biosensors

| | | | | |
|---|---|---|---|---|
| 1-butanol | Fatty alcohol, fuel | BmoR | Allosteric TF | [1] |
| Acrylate | Plastic precursor | AcuR | Allosteric TF | [2**] |
| Adipate | Dicarboxylic acid | PcaR | Allosteric TF | [1] |
| B12 | Vitamin | BtuB | Riboswitch | [3] |
| Benzoate, naphthalene | Aromatics | NahR | Allosteric TF | [4] |
| Erythromycin | Macrolide | MphR | Allosteric TF | [5] |
| Fatty acids | Fatty acid | FadR | Allosteric TF | [6] |
| Fatty acids | Fatty acid | | GPCR | [7] |
| Glucarate | Feedstock | CdaR | Allosteric TF | [8] |
| Lysine | Amino acid | LysR | Allosteric TF | [9] |
| Muconate | Dicarboxylic acid | BenM | Allosteric TF | [10] |
| NADPH | Redox | SoxR | Allosteric TF | [11] |
| Naringenin | Flavonoid | TtgR | Allosteric TF | [12] |
| Octane | Alkane | AlkS | Allosteric TF | [13] |
| Succinate | Dicarboxylic acid | DcuR | Two-component | [1] |
| Tetracyclines | Polyketides | TetR | Allosteric TF | [14] |

Engineered biosensors

| | | | | |
|---|---|---|---|---|
| 3,4-dihydroxybenzoate | Aromatic | PobR | Allosteric TF | [15] |
| Biphenyl, nitrotoluenes | Aromatics | XylR | Allosteric TF | [16] |
| Mevalonate | Isoprenoid precursor | AraC | Allosteric TF | [17] |
| Pyruvate | Alpha-keto acid | De novo | FRET | [18] |
| Theophylline | Alkaloid | De novo | Riboswitch | [19] |
| Thiamine-pp | Vitamin | De novo | Riboswitch | [20, 21] |
| Trehalose-6-p | Sugar | De novo | FRET | [22] |
| Triacetic acid lactones | Feedstock | AraC | Allosteric TF | [23] |
| Vanillin | Aromatic, flavoring | QacR | Allosteric TF | [24] |
| Zn²⁺ | Ion | De novo | FRET | [25] |

TABLE 3
Examples of biosensor-mediated high-
throughput metabolic engineering

| | Biosensor | Titer fold | | | |
|---|---|---|---|---|---|
| Molecule | mode | improvement | Throughput | Year | Reference |
| naringenin | selection | 36 | 10⁹ | 2014 | [26] |
| glucarate | selection | 22 | 10⁹ | 2014 | [26] |
| lysine | screen | 37 | 10⁸ | 2013 | [27] |
| histidine | screen | >40 | 10⁸ | 2013 | [27] |
| arginine | screen | 87 | 10⁸ | 2013 | [27] |
| triacetate | screen | 20 | 10⁴ | 2013 | [28] |
| lactone | | | | | |
| mevalonate | screen | 3.8 | 10⁵ | 2011 | [29] |
| butanol | screen | 1.4 | 10³ | 2010 | [30] |

### REFERENCES

1. Dietrich, J. A., et al., Transcription factor-based screens and synthetic selections for microbial small-molecule biosynthesis. ACS Synth Biol, 2013. 2(1): p. 47-58.
2. Rogers, J. K., et al., Synthetic biosensors for precise gene control and real-time monitoring of metabolites, in Nucleic Acids Res. 2015. p. 7648-7660.
** This study combines biosensors for four plastic precursors (acrylate, 3-hydroxypropionate, muconate and glucarate) with their respective metabolic pathways to demonstrate fluorescent biosensors can be used to track metabolite production in real-time. A small screen is used to demonstrate biosensor-based culture condition optimization for the production of 3-hydroxypropionate.
3. Fowler, C. C., E. D. Brown, and Y. Li, Using a riboswitch sensor to examine coenzyme B(12) metabolism and transport in E. coli, in Chem. Biol. 2010. p. 756-765.
4. van Sint Fiet, S., J. B. van Beilen, and B. Witholt, Selection of biocatalysts for chemical synthesis, in Proc. Natl. Acad. Sci. U.S.A. 2006. p. 1693-1698.
5. Zheng, J., et al., Structure and function of the macrolide biosensor protein, MphR(A), with and without erythromycin, in J. Mol. Biol. 2009. p. 1250-1260.
6. Zhang, F. Z., J. M. Carothers, and J. D. Keasling, Design of a dynamic sensor-regulator system for production of chemicals and fuels derived from fatty acids. Nature Biotechnology, 2012. 30(4): p. 354-U166.
7. Mukherjee, K., S. Bhattacharyya, and P. Peralta-Yahya, GPCR-Based Chemical Biosensors for Medium-Chain Fatty Acids. ACS Synth Biol, 2015. 4(12): p. 1261-9.
8. Monterrubio, R., et al., A common regulator for the operons encoding the enzymes involved in D-galactarate, D-glucarate, and D-glycerate utilization in Escherichia coli. J Bacteriol, 2000. 182(9): p. 2672-4.
9. Schell, M. A., Molecular biology of the LysR family of transcriptional regulators, in Annu. Rev. Microbiol. 1993. p. 597-626.
10. Craven, S. H., et al., Inducer responses of BenM, a LysR-type transcriptional regulator from Acinetobacter baylyi ADP1, in Mol. Microbiol. 2009. p. 881-894.
11. Siedler, S., et al., SoxR as a single-cell biosensor for NADPH-consuming enzymes in Escherichia coli. ACS Synth Biol, 2014. 3(1): p. 41-7.
12. Terán, W., et al., Antibiotic-dependent induction of Pseudomonas putida DOT-T1E TtgABC efflux pump is mediated by the drug binding repressor TtgR, in Antimicrob. Agents Chemother. 2003. p. 3067-3072.
13. Canosa, I., L. Yuste, and F. Rojo, Role of the alternative sigma factor sigmaS in expression of the AlkS regulator of the Pseudomonas oleovorans alkane degradation pathway, in J. Bacteriol. 1999. p. 1748-1754.
14. Lutz, R. and H. Bujard, Independent and tight regulation of transcriptional units in Escherichia coli via the LacR/O, the TetR/O and AraC/I1-I2 regulatory elements, in Nucleic Acids Res. 1997. p. 1203-1210.
15. Jha, R. K., et al., Rosetta comparative modeling for library design: Engineering alternative inducer specificity in a transcription factor. Proteins, 2015.
16. Garmendia, J., et al., A la carte transcriptional regulators: unlocking responses of the prokaryotic enhancer-binding protein XylR to non-natural effectors, in Mol. Microbiol. 2001. p. 47-59.
17. Tang, S.-Y. and P. C. Cirino, Design and application of a mevalonate-responsive regulatory protein, in Angew. Chem. Int. Ed. Engl. 2011. p. 1084-1086.
18. San Martin, A., et al., Imaging mitochondrial flux in single cells with a FRET sensor for pyruvate. PLoS One, 2014. 9(1): p. e85780.
19. Lynch, S. A. and J. P. Gallivan, A flow cytometry-based screen for synthetic riboswitches, in Nucleic Acids Res. 2009. p. 184-192.
20. Muranaka, N., K. Abe, and Y. Yokobayashi, Mechanism-guided library design and dual genetic selection of synthetic OFF riboswitches. Chembiochem, 2009. 10(14): p. 2375-81.
21. Muranaka, N., et al., An efficient platform for genetic selection and screening of gene switches in Escherichia coli. Nucleic Acids Res, 2009. 37(5): p. e39.
22. Peroza, E. A., et al., A genetically encoded Forster resonance energy transfer sensor for monitoring in vivo trehalose-6-phosphate dynamics. Anal Biochem, 2015. 474: p. 1-7.
23. Tang, S.-Y., et al., Screening for enhanced triacetic acid lactone production by recombinant Escherichia coli expressing a designed triacetic acid lactone reporter., in J. Am. Chem. Soc. 2013. p. 10099-10103.
24. de Los Santos, E. L., et al., Engineering Transcriptional Regulator Effector Specificity Using Computational Design and In Vitro Rapid Prototyping: Developing a Vanillin Sensor. ACS Synth Biol, 2015.
25. Vinkenborg, J. L., et al., Genetically encoded FRET sensors to monitor intracellular Zn2+ homeostasis. Nat Methods, 2009. 6(10): p. 737-40.
26. Raman, S., et al., Evolution-guided optimization of biosynthetic pathways. Proc Natl Acad Sci USA, 2014. 111(50): p. 17803-8.
27. Schendzielorz, G., et al., Taking control over control: use of product sensing in single cells to remove flux control at key enzymes in biosynthesis pathways. ACS Synth Biol, 2014. 3(1): p. 21-9.
28. Tang, S. Y., et al., Screening for enhanced triacetic acid lactone production by recombinant Escherichia coli expressing a designed triacetic acid lactone reporter. J Am Chem Soc, 2013. 135(27): p. 10099-103.
29. Tang, S. Y. and P. C. Cirino, Design and application of a mevalonate-responsive regulatory protein. Angew Chem Int Ed Engl, 2011. 50(5): p. 1084-6.
30. Dietrich, J. A., A. E. McKee, and J. D. Keasling, High-Throughput Metabolic Engineering: Advances in Small-Molecule Screening and Selection. Annual Review of Biochemistry, Vol 79, 2010. 79: p. 563-590.

TABLE 4
Sequence of regulator proteins and cognate promoter/operators.

| | Promoter/ | |
|---|---|---|
| | Operator | |
| Regulator | Sequence | Regulator Sequence |
| acuR | GCTTCACAACCGCACT | ATGCCGCTGACCGACACCCCGCCGTCTGTTCCGCAGAAAC |
| | TGATTTAATAGACCAT | CGCGTCGTGGTCGTCCGCGTGGTGCTCCGGACGCTTCTCT |
| | ACCGTCTATTATTTCTG | GGCTCACCAGTCTCTGATCCGTGCTGGTCTGGAACACCTGA |
| | G | CCGAAAAAGGTTACTCTTCTGTTGGTGTTGACGAAATCCTG |
| | (SEQ ID NO: 1) | AAAGCTGCTCGTGTTCCGAAAGGTTCTTTCTACCACTACTT |
| | | CCGTAACAAAGCTGACTTCGGTCTGGCTCTGATCGAAGCTT |
| | | ACGACACCTACTTCGCTCGTCTCCTCGACCAGGCGTTCCTG |
| | | GACGGTTCGCTGGCTCCGCTGGCTCGTCTGCGTCTGTTCAC |
| | | CCGTATGGCTGAAGAAGGTATGGCTCGTCACGGTTTCCGT |
| | | CGTGGTTGCCTGGTTGGTAACCTGGGTCAGGAAATGGGTG |
| | | CTCTGCCGGACGACTTCCGTGCTGCTCTGATCGGTGTTCTG |
| | | GAAACCTGGCAGCGTCGTACCGCTCAGCTGTTCCGTGAAG |
| | | CTCAGGCTTGCGGTGAACTGTCTGCTGACCACGACCCGGA |
| | | CGCTCTGGCTGAAGCTTTCTGGATCGGTTGGGAAGGTGCT |
| | | ATCCTGCGTGCTAAACTGGAACTGCGTCCGGACCCGCTGC |
| | | ACTCTTTCACCCGTACCTTCGGTCGTCACTTCGTTACCCGTA |
| | | CCCAGGAATAA |
| | | (SEQ ID NO: 5) |
| araC | AGAAACCAATTGTCCA | ATGGCTGAAGCGCAAAATGATCCCCTGCTGCCGGGATACT |
| | TATTGCATCAGACATT | CGTTTAACGCCCATCTGGTGGCGGGTTTAACGCCGATTGA |
| | GCCGTCACTGCGTCTT | GGCCAACGGTTATCTCGATTTTTTTATCGACCGACCGCTGG |
| | TTACTGGCTCTTCTCGC | GAATGAAAGGTTATATTCTCAATCTCACCATTCGCGGTCAG |
| | TAACCCAACCGGTAAC | GGGGTGGTGAAAAATCAGGGACGAGAATTTGTCTGCCGA |
| | CCCGCTTATTAAAAGC | CCGGGTGATATTTTGCTGTTCCCGCCAGGAGAGATTCATCA |
| | ATTCTGTAACAAAGCG | CTACGGTCGTCATCCGGAGGCTCGCGAATGGTATCACCAG |
| | GGACCAAAGCCATGAC | TGGGTTTACTTTCGTCCGCGCGCCTACTGGCATGAATGGCT |
| | AAAAACGCGTAACAAA | TAACTGGCCGTCAATATTTGCCAATACGGGTTTCTTTCGCC |
| | AGTGTCTATAATCACG | CGGATGAAGCGCACCAGCCGCATTTCAGCGACCTGTTTGG |
| | GCAGAAAAGTCCACAT | GCAAATCATTAACGCCGGGCAAGGGGAAGGGCGCTATTC |
| | TGATTATTTGCACGGC | GGAGCTGCTGGCGATAAATCTGCTTGAGCAATTGTTACTG |
| | GTCACACTTTGCTATGC | CGGCGCATGGAAGCGATTAACGAGTCGCTCCATCCACCGA |
| | CATAGCATTTTTATCCA | TGGATAATCGGGTACGCGAGGCTTGTCAGTACATCAGCGA |
| | TAAGATTAGCGGATCC | TCACCTGGCAGACAGCAATTTTGATATCGCCAGCGTCGCAC |
| | TACCTGACGCTTTTTAT | AGCATGTTTGCTTGTCGCCGTCGCGTCTGTCACATCTTTTCC |
| | CGCAACTCTCTACTGTT | GCCAGCAGTTAGGGATTAGCGTCTTAAGCTGGCGCGAGG |
| | TCTCCATACCCGCTTTC | ACCAACGCATTAGTCAGGCGAAGCTGCTTTTGAGCACTAC |
| | ATATCTTTCACTTTTTTT | CCGGATGCCTATCGCCACCGTCGGTCGCAATGTTGGTTTTG |
| | GGGCTAAC | ACGATCAACTCTATTTCTCGCGAGTATTTAAAAAATGCACC |
| | (SEQ ID NO: 10) | GGGGCCAGCCCGAGCGAGTTTCGTGCCGGTTGTGAAGAA |
| | | AAAGTGAATGATGTAGCCGTCAAGTTGTCAtaa |
| | | (SEQ ID NO: 11) |
| cdaR | ATGCTGTTGATTGACG | ATGGCTGGCTGGCATCTTGATACCAAAATGGCGCAGGATA |
| | CCAGTGAGAACCCGGA | TCGTGGCACGTACCATGCGCATCATCGATACCAATATCAAC |
| | ACCGGAAACGGAATCA | GTAATGGATGCCCGTGGGCGAATTATCGGCAGCGGCGATC |
| | AATCCGTGGGTCGAAC | GTGAGCGTATTGGTGAATTGCACGAAGGTGCATTGCTGGT |
| | AGTGGGGCACGCTGTT | ACTTTCACAGGGACGAGTCGTCGATATCGATGACGCGGTA |
| | GTCCTGATATGTTCAG | GCACGTCATCTGCACGGTGTGCGGCAGGGGATTAATCTAC |
| | CGAGCGGTAAATGTCG | CGTTACGGCTGGAAGGTGAAATTGTCGGCGTAATTGGCCT |
| | TTTTAGCGGTGCTGAA | GACAGGTGAACCAGAGAATCTGCGTAAATATGGCGAACT |
| | TCGAATCTTTTCAGGC | GGTCTGCATGACGGCTGAAATGATGCTGGAACAGTCGCG |
| | AAATGCCAGTAAAAAC | GTTGATGCACTTGTTGGCGCAGGATAGCCGTTTGCGGGAA |
| | TGCTTCATAGCGCGGA | GAACTGGTGATGAACCTGATTCAGGCAGAGGAGAATACTC |
| | TTTTTACTGGCGTTTGC | CCGCACTTACTGAATGGGCGCAACGGCTGGGGATCGATCT |
| | CTGGAGTCAAGCGATC | CAATCAACCGCGAGTGGTGGCTATTGTTGAGGTCGACAGC |
| | CATTTCATACTCTTCTT | GGTCAGCTTGGCGTGGACAGCGCAATGGCGGAGTTACAA |
| | TATTTCTTCGTTTTAAC | CAACTGCAAAACGCGCTGACTACGCCCGAGCGTAATAATC |
| | CCTTCCTTTCTTGTTCTT | TGGTGGCGATTGTCTCGCTAACCGAAATGGTGGTGTTGAA |
| | GTTTTCATTTCCGTGAA | ACCGGCGTTGAACTCTTTTGGGCGCTGGGATGCAGAAGAT |
| | GTGGATTCCACCGTCC | CATCGTAAGCGAGTTGAACAACTGATTACCCGCATGAAAG |
| | AGGGCTAATGCCAAAA | AGTACGGCCAGCTGCGTTTTCGCGTTTCACTGGGCAACTAT |
| | TCGGGCCTCATTGAAC | TTTACCGGTCCTGGCAGTATTGCCCGATCCTATCGTACGGC |
| | GCATTAATGTTGTGTT | GAAAACGACGATGGTGGTGGGTAAACAGCGGATGCCAGA |
| | GTTGCACGGTGAGCCG | AAGTCGCTGCTATTTTTATCAGGATCTGATGTTACCTGTGT |
| | CTATGGCGCGCTTTTTA | TACTCGACAGTTTGCGTGGCGACTGGCAGGCCAACGAACT |
| | TACTGCTATTGCCAGA | GGCGCGACCGCTGGCGCGGCTGAAAACGATGGACAATAA |
| | TATAAACACGCGCCGT | CGGCTTGCTGCGACGAACGCTGGCGGCGTGGTTTCGCCAC |
| | ATTCGGCGAACGACCT | AATGTGCAACCGCTGGCAACGTCAAAGGCGTTGTTTATTC |
| | ATAAAAACGGCAAAAA | ATCGTAATACCCTGGAGTATCGGCTTAATCGTATATCGGAA |
| | ACACCCTACGTCACCTC | CTGACCGGGCTTGATTTGGGCAATTTTGATGACAGGTTGC |
| | TGATTTCCTGGCGATG | TGCTGTATGTGGCGTTACAACTGGATGAAGAGCGGtag |
| | TCGCAGTCCAGAGTGA | (SEQ ID NO: 13) |
| | GCGTGGCTAACGCGAA | |
| | TTTTCAGGAGTGCAAC | |
| | A | |
| | (SEQ ID NO: 12) | |
| mphR | GGATTGAATATAACCG | ATGCCGCGTCCGAAACTGAAATCTGACGACGAAGTTCTGG |
| | ACGTGACTGTTACATTT | AAGCGGCGACCGTTGTTCTGAAACGTTGCGGTCCGATCGA |
| | AGGTGGCTAAACCCGT | ATTCACCCTGTCTGGTGTTGCGAAAGAAGTTGGTCTGTCTC |
| | CAA | GTGCGGCGCTGATCCAGCGTTTCACCAACCGTGACACCCT |
| | (SEQ ID NO: 14) | GCTGGTTCGTATGATGGAACGTGGTGTTGAACAGGTTCGT |
| | | CACTACCTGAACGCGATCCCGATCGGTGCGGGTCCGCAGG |
| | | GTCTGTGGGAATTCCTGCAGGTTCTGGTTCGTTCTATGAAC |
| | | ACCCGTAACGACTTCTCTGTTAACTACCTGATCTCTTGGTAC |
| | | GAACTGCAGGTTCCGGAACTGCGTACCCTGGCGATCCAGC |
| | | GTAACCGTGCGGTTGTTGAAGGTATCCGTAAACGTCTGCC |
| | | GCCGGGTGCGCCGGCGGCGGCGGAACTGCTGCTGCACTC |
| | | TGTTATCGCGGGTGCGACCATGCAGTGGGCGGTTGACCCG |
| | | GACGGTGAACTGGCGGACCACGTTCTGGCGCAGATCGCG |
| | | GCGATCCTGTGCCTGATGTTCCCGGAACACGACGACTTCCA |
| | | GCTGCTGCAGGCGCACGCGTAA |
| | | (SEQ ID NO: 15) |
| tetR | TCGAGTCCCTATCAGT | ATGTCTCGTTTAGATAAAAGTAAAGTGATTAACAGCGCATT |
| | GATAGAGATTGACATC | AGAGCTGCTTAATGAGGTCGGAATCGAAGGTTTAACAACC |
| | CCTATCAGTGATAGAG | CGTAAACTCGCCCAGAAGCTAGGTGTAGAGCAGCCTACAT |
| | ATACTGAGCACATCAG | TGTATTGGCATGTAAAAAATAAGCGGGCTTTGCTCGACGC |
| | CAGGACGCACTGACCG | CTTAGCCATTGAGATGTTAGATAGGCACCATACTCACTTTT |
| | AATTCATTAAA | GCCCTTTAGAAGGGGAAAGCTGGCAAGATTTTTTACGTAA |
| | (SEQ ID NO: 16) | TAACGCTAAAAGTTTTAGATGTGCTTTACTAAGTCATCGCG |
| | | ATGGAGCAAAAGTACATTTAGGTACACGGCCTACAGAAAA |
| | | ACAGTATGAAACTCTCGAAAATCAATTAGCCTTTTTATGCC |
| | | AACAAGGTTTTTCACTAGAGAATGCATTATATGCACTCAGC |
| | | GCAGTGGGGCATTTTACTTTAGGTTGCGTATTGGAAGATC |
| | | AAGAGCATCAAGTCGCTAAAGAAGAAAGGGAAACACCTA |
| | | CTACTGATAGTATGCCGCCATTATTACGACAAGCTATCGAA |
| | | TTATTTGATCACCAAGGTGCAGAGCCAGCCTTCTTATTCGG |
| | | CCTTGAATTGATCATATGCGGATTAGAAAAACAACTTAAAT |
| | | GTGAAAGTGGGTCTTAA |
| | | (SEQ ID NO: 17) |
| ttgR | CACCCAGCAGTATTTA | ATGGTGCGTCGCACCAAAGAAGAAGCACAGGAAACGCGT |
| | CAAACAACCATGAATG | GCGCAGATTATCGAAGCGGCCGAACGCGCGTTTTATAAAC |
| | TAAGTATATTCCTTAGC | GTGGTGTGGCACGTACCACGCTGGCAGATATTGCAGAACT |
| | AA | GGCAGGTGTTACCCGCGGTGCAATCTACTGGCATTTCAAC |
| | (SEQ ID NO: 18) | AATAAAGCCGAACTGGTTCAGGCACTGCTGGATTCTCTGC |
| | | ACGAAACGCATGATCACCTGGCCCGTGCAAGCGAATCTGA |
| | | AGATGAACTGGACCCGCTGGGCTGCATGCGCAAACTGCTG |
| | | CTGCAGGTGTTTAACGAACTGGTTCTGGATGCACGTACCC |
| | | GTCGCATTAATGAAATCCTGCATCACAAATGCGAATTTACG |
| | | GATGATATGTGTGAAATTCGTCAGCAGCGCCAGAGCGCCG |
| | | TGCTGGATTGTCATAAAGGTATCACCCTGGCACTGGCAAA |
| | | CGCAGTTCGTCGCGGTCAGCTGCCGGGTGAACTGGATGTG |
| | | GAACGCGCAGCGGTTGCGATGTTTGCCTATGTGGATGGCC |
| | | TGATTGGTCGTTGGCTGCTGCTGCCGGATAGTGTTGATCT |
| | | GCTGGGCGATGTGGAAAAATGGGTTGATACCGGTCTGGA |
| | | TATGCTGCGTCTGAGCCCGGCGCTGCGCAAATAA |
| | | (SEQ ID NO: 19) |

TABLE 5
Sequence of MIOX orthologs

| MIOX | |
|---|---|
| Variant | Sequence |
| Candida | ATGGTAAACAAGGTCGGTAAATCTACTCTCGATAAGAGCACAAACCTAG |
| albicans | ATAAATCCAAAGGGAATATATTAGAGAAACTAGATGATGATATACTTCAT |
| | GTCAATAGAATTCGAGGCTCTTTAACTAACAAAACTCCAATCACCAAAAC |
| | CCATTCGATAGATGATGAGCTTAAACTAGAAGAACAATCAGAAACTGCC |
| | GCCGATGAAAATTGGCAAATAGCATCGGAATATTATAAAAACATAGACA |
| | CGAAGGCTTTCCGCCAATATGAATTAGCTTGTGATAGAGTCAAACAGTTT |
| | TATGAAGAACAACATGAAAAACAAACCGTGGCGTATAATATTCAAGCAA |
| | GAATTAATTTCAAAACTAAAACAAGAGCAAGAATGACAGTTTGGGAAGG |
| | ACTAGAGAAATTAAACAAATTGTTAGATGATTCTGATCCCGACACCGAAT |
| | TGTCACAAATAGATCATGCATTACAGACGGCAGAAGCTATACGGCGAGA |
| | TGGGAAACCACGATGGTTTCAATTAGTTGGGTTGATTCATGATTTAGGGA |
| | AATTACTATATTTTTTTGATTCTCGTGGTCAATGGGATGTAGTGGGTGATA |
| | CTTTCCCTGTTGGTTGTAAATTCCTGAAACGGATTATTTTCCCTGATAGTTT |
| | TAAAAATAATCCAGATTTCCTAAATCCATTGTATAATACCAAATATGGCAT |
| | ATATTCAAAACATTGTGGATTAGATAAAGTCATGTTGAGTTGGGGTCATG |
| | ATGAGTATATGTATCATGTTGCGAAAAAGAATTCGACATTACCACCGGAA |
| | GCATTGGCAATGATAAGGTATCATTCATTTTATCCTTGGCATCAAGAATTG |
| | GCATATAGTTATTTAATGGATGAGCATGATAAAGAGATGTTGAAAGCAG |
| | TCAAAGCTTTCAATTCCTATGATTTATATTCCAAGATAGATCAACAGTATG |
| | ATGTTGAAGAGTTGAAACCATATTACCTAGAGTTGATTGATGAGTTTTTC |
| | CCAAATAAAGTAATTGATTTTTAA |
| | (SEQ ID NO: 20) |
| Francisella | ATGAGTCAGACCGTGGAAAACACGTTTGGCGAATTTCGTAACTACACCG |
| sp. | ATAGCAAATTCCAGGATCGTGTGGAACGCACGTACAAAGATATGCACATT |
| TX077308 | AACCAGAATCTGGAATACGTTACCCAGATGAAAGATAAATACTTCAAACT |
| | GGATCTGGGTAAAATGGATGTGTACGAAGTTTTCAAACTGCTGGAAAAC |
| | GTTCATGATGAAAGCGATCCGGATAATGATCTGCCGCAGATCGAACACG |
| | CATATCAGACCGCGGAAGCCTGCCAGAACAAATTCCTGAAATCTGATACG |
| | GAACTGCGCGAAAATGCGCTGATTCGTAGTATCTTTCGCGATCATGAATG |
| | GCAGAGCATTCCGAAAATCTGGCAGGATTTCTATACCAAAAAACAGAGTC |
| | TGGGCAATCTGTACAGCCATATTAAAGATTGGTCTTGGTTTCCGCTGGTT |
| | GGCTTCGTTCACGATCTGGGTAAAATCATGACCCTGCCGGAATATGGTCA |
| | GCTGCCGCAGTGGAGCACCGTGGGTGATACGTACCCGATTGCCTGCCCG |
| | TTTGCAAGCGCGAACGTGTTTTCTCACCGTGAATTTGTTAAAGATTCTAAA |
| | GATTACAACAATTACAATACCGAAAGTGAAACGCATTATGGCAAATACGA |
| | GAAAAAATGTGGTTTCGATAACGTGGATATGAGCTTCGGTCACGATGAA |
| | TACATCTACAAAGTTTTCGAACAGGGCAGCGATATCCCGTATGAAGGTCT |
| | GTACCTGCTGCGCTATCATTCTTTCTACCCGTGGCACACCCCGCAGACGG |
| | GCGGTCATGCGTATCAGGAACTGGCCAACGAAAAAGATTGGCTGCTGCT |
| | GCCGCTGCTGAAAGCCTTTCAGAAAGCGGATCTGTATTCTAAACTGCCGG |
| | AACTGCCGCCGAAAGAAGTGCTGGAGAAAAAATACAAAAGTCTGCTGGA |
| | TAAATGGGTTCCGAACAAGAAAATTAACTGGTAA |
| | (SEQ ID NO: 21) |
| Flavobacterium | ATGAAAAAGCATATAGACACAGACAATCCGTTGAAAAATTTAGATGAGT |
| johnsoniae | GGGAAGATGATTTGTTAATGCGATATCCTGACCCTTCTGAAGTAAATGAA |
| | AGTTTAAAAGAAAAGCAGAAAGAAGAATTTAGAAATTATGTCGATTCTG |
| | AAAGAGTAGAAACGGTAAAAGAATTTTACAGGATAAACCATACCTACCA |
| | AACTTATGACTTTGTATGCAGTAAAGAACAAGAATTTCTGCAATTTAATA |
| | GAAAAGAAATGTCAATCTGGGAAGCTGTCGAGTTTTTAAACACGCTTGTA |
| | GACGACAGTGACCCAGATATTGACTTAGACCAGACACAGCACCTTTTACA |
| | GACTTCAGAAGCCATTCGTGCTGATGGTCATCCGGATTGGTTTGTACTGA |
| | CAGGTTTCATTCACGATTTGGGTAAAGTTTTATGCTTATTTGGAGAACCGC |
| | AATGGGCAGTCGTTGGCGATACTTTTCCGGTTGGCTGTGCGTATTCGGAT |
| | AAAATTGTGTATTCAGAATTTTTTAAAGAAAATCCGGATTATACAGATGA |
| | GAGATTCAATACTAAACTAGGAATCTACACTGAAAACTGCGGATTAGATA |
| | ACGTAAAAATGAGCTGGGGTCATGACGAATATTTGTATCAGATTATGAA |
| | AGATTATTTACCGGATCCTGCTTTATACATGATTCGTTATCACTCTTTTTAT |
| | TCGCAGCATAAAGAAAATGCGTATGCACATTTAATGAATGAAAAAGACA |
| | TCGAAATGTTTGACTGGGTTCGAAAATTCAATCCGTACGATTTGTATACA |
| | AAGGCTCCTGTAAAACCAGATGTTCAGGCATTACTTCCTTATTATAAAGA |
| | ATTAGTTGCTAAATATTTGCCTGAAAAATTGAAGTTTTAA |
| | (SEQ ID NO: 22) |
| Mus musculus | ATGAAAGTGGATGTTGGCCCGGACCCGAGCCTGGTTTACCGCCCGGATG |
| | TGGACCCGGAAATGGCAAAAAGCAAAGATTCGTTTCGTAACTACACCAG |
| | TGGCCCGCTGCTGGATCGTGTTTTTACCACGTATAAACTGATGCATACCC |
| | ACCAGACGGTTGACTTTGTCAGCCGTAAACGCATTCAATATGGCGGTTTC |
| | TCTTACAAGAAAATGACCATCATGGAAGCGGTGGGCATGCTGGATGACC |
| | TGGTTGATGAATCAGATCCGGACGTCGATTTTCCGAATTCGTTTCATGCG |
| | TTCCAGACGGCCGAAGGTATTCGCAAAGCCCACCCGGACAAAGATTGGT |
| | TCCATCTGGTCGGCCTGCTGCACGATCTGGGTAAAATCATGGCACTGTGG |
| | GGTGAACCGCAGTGGGCTGTGGTTGGTGATACCTTTCCGGTGGGTTGCC |
| | GTCCGCAAGCAAGTGTCGTGTTTTGTGACTCCACCTTCCAGGACAACCCG |
| | GATCTGCAAGACCCGCGCTATTCAACGGAACTGGGCATGTACCAGCCGC |
| | ATTGCGGTCTGGAAAACGTGCTGATGTCGTGGGGTCACGATGAATACCT |
| | GTACCAGATGATGAAATTCAACAAATTCAGCCTGCCGTCTGAAGCCTTCT |
| | ACATGATCCGTTTCCATAGTTTCTACCCGTGGCACACCGGCGGTGATTATC |
| | GCCAGCTGTGCTCCCAGCAAGACCTGGATATGCTGCCGTGGGTGCAAGA |
| | ATTCAACAAATTCGATCTGTACACGAAATGTCCGGATCTGCCGGACGTTG |
| | AATCTCTGCGTCCGTACTACCAAGGTCTGATTGATAAATACTGTCCGGGC |
| | ACCCTGTCGTGGTAA |
| | (SEQ ID NO: 23) |

### Example V

### In Vitro Screening Using Addition of Purified Sensor Protein

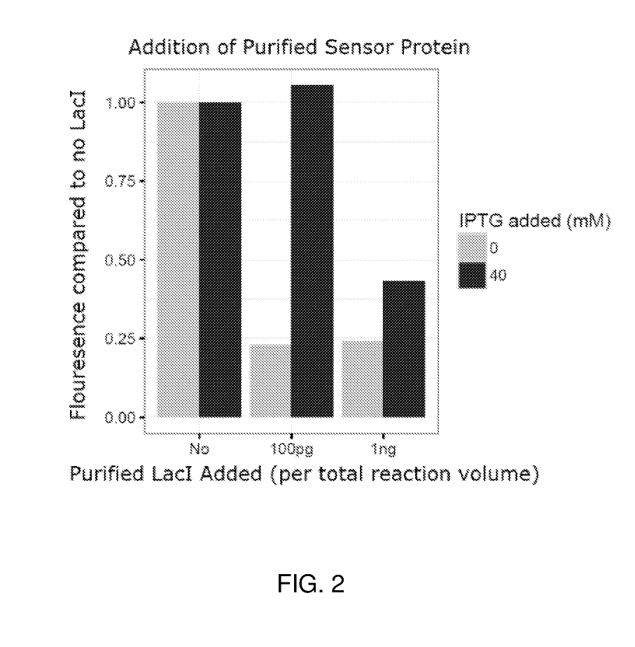

A plasmid containing a T7 promoter, lac operator/binding site, RBS, and GFP coding region was constructed (pET-minus_lacI) and prepared for in vitro transcription and translation reactions. The addition of purified LacI represses the constitutive plasmid production of GFP at 100 pg/μL and 1 ng/μL levels. Fluorescent measurement of GFP production after induction of the sensor by the addition of IPTG (40 mM) shows increased GFP production. The addition of purified sensor allows for fine-tuning of repression levels present in each reaction well and prevents cofounding effects of library variants impacting in vitro sensor production. (FIG. 2)

### Methods:

A 100 ng/μL dilution of purified LacI protein (Novoprotein Cat No. CG57) was created in a buffer containing 20 mM Tris, 300 mM NaCl, and 5 mM DTT. Serial dilutions were performed to obtain concentrations of 1 ng/μL and 0.1 ng/μL. The cell-free reactions used 4 μL of S30 Premix Plus (Promega kit Cat No. L1110). Then, RNase inhibitor (ThermoFisher Cat No. N8080119) was added to a final reaction concentration of 0.05% to each of the reaction tubes. Then, 3.6 μL of T7-S30 Circular Extract (Promega kit Cat No. L1110) was added to all tubes, followed by 2 μL of purified pET-GFP plasmid DNA at 99.3 μg/μL and 0.4 μL of water.

For induction and response measurement, 1 μL of 400 μM IPTG was added to half of the tubes, for a final IPTG concentration of 40 μM. Finally, 1 μL of the LacI dilutions at each of the indicated concentrations was added.

Reactions were assembled on ice, then vortexed briefly, spun down, and incubated at 37° C. overnight. After incubation, 10 μL of each reaction was put into a well of a black, clear/flat-bottom, 384-well-plate, and fluorescence was measured on the Biotek Synergy Neo using 485 nm excitation and 528 nm emission wavelengths.

Mutant identification: Each well of a reaction plate contains a library member with genetic variation affecting biosynthesis of a target molecule. Exogenous addition of purified sensor allows for fine-tuning of repression levels present in each reaction well, permitting selection of library members showing exceptional sensor response across multiple, controlled sensor concentration levels.

LOCUS pET_minus_lacI 4813 bp ds-DNA linear 15-JUL-2016
DEFINITION.

| FEATURES | Location/Qualifiers |
|---|---|
| misc_feature | 856 . . . 858 |
| | /label = “split2” |
| protein_binding | 412 . . . 432 |
| | /label = “lacO” |
| misc_feature | 1024 . . . 1026 |
| | /label = “split5” |
| misc_feature | 1184 . . . 1189 |
| | /label = “PstI” |
| misc_feature | 478 . . . 1221 |
| | /label = “stGFP” |
| misc_feature | 658 . . . 660 |
| | /label = “split1” |
| misc_feature | 1237 . . . 1242 |
| | /label = “AvaI” |
| CDS | complement(4051 . . . 4242) |
| | /label = “ROP” |
| protein_binding | 1249 . . . 1293 |
| | /label = “S-Tag” |
| misc_feature | 736 . . . 741 |
| | /label = “NdeI” |
| misc_feature | 3863 . . . 3868 |
| | /label = “ApaLI” |
| misc_feature | 925 . . . 927 |
| | /label = “split3” |
| misc_feature | 476 . . . 481 |
| | /label = “NcoI” |
| misc_feature | 1231 . . . 1236 |
| | /label = “KpnI” |
| rep_origin | 1430 . . . 1877 |
| | /label = “f1 ori” |
| misc_feature | 976 . . . 978 |
| | /label = “split4” |
| terminator | 1345 . . . 1392 |
| | /label = “T7 terminator” |
| CDS | complement(1999 . . . 2859) |
| | /label = “Amp” |
| misc_feature | 1171 . . . 1176 |
| | /label = “EcoRI” |
| misc_feature | 3363 . . . 3368 |
| | /label = “ApaLI” |
| misc_feature | 965 . . . 970 |
| | /label = “EagI” |
| misc_feature | 2739 . . . 2744 |
| | /label = “ApaLI” |
| misc_feature | 391 . . . 407 |
| | /label = “T7 promoter” |
| misc_feature | 1090 . . . 1092 |
| | /label = “split6” |
| misc_feature | 369 . . . 374 |
| | /label = “ClaI” |
| misc_feature | 508 . . . 513 |
| | /label = “NheI” |
| RBS | 465 . . . 470 |
| | /label = “RBS-1” |
| rep_origin | 3620 . . . 3620 |
| | /label = “ColE1, pBR322 ori” |
| misc_feature | 1237 . . . 1242 |
| | /label = “XhoI” |

```
ORIGIN (SEQ ID NO: 24)
      1 attcaccacc ctgaattgac tctcttccgg gcgctatcat gccataccgc gaaaggtttt
    61 gcgccattcg atggtgtccg ggatctcgac gctctccctt atgcgactcc tgcattagga
  121 agcagcccag tagtaggttg aggccgttga gcaccgccgc cgcaaggaat ggtgcatgca
  181 aggagatggc gcccaacagt cccccggcca cggggcctgc caccataccc acgccgaaac
  241 aagcgctcat gagcccgaag tggcgagccc gatcttcccc atcggtgatg tcggcgatat
  301 aggcgccagc aaccgcacct gtggcgccgg tgatgccggc cacgatgcgt ccggcgtaga
  361 ggatcgagat cgatctcgat cccgcgaaat taatacgact cactataggg gaattgtgag
  421 cggataacaa ttcccctcta gaaataattt tgtttaactt taagaaggag atataccatg
  481 ggtcatcacc accaccatca cggtggcgct agcaaaggtg aagagctgtt tacgggtgta
  541 gtaccgatct tagtggaatt agacggcgac gtgaacggtc acaaatttag cgtgcgcggc
  601 gaaggcgaag gtgacgctac caatggtaaa ttgaccctga agtttatttg cacaacaggc
  661 aaattacccg ttccgtggcc caccttagtg accaccctga cctatggcgt tcagtgcttc
  721 agtcgttacc cagatcatat gaaacaacac gattttttca aatcagccat gcctgaagga
  781 tatgttcaag agcgtacaat cagcttcaag gacgatggca cctataaaac gcgtgcggaa
  841 gtgaaatttg aaggcgacac attagtaaac cgtatcgaac tgaaaggtat cgacttcaaa
  901 gaagacggca acattttagg ccataagctg gaatataact ttaattctca taacgtgtat
  961 attacggccg ataaacagaa aaacggtatc aaggcaaatt tcaaaattcg ccataacgtg
1021 gaagacggca gcgttcaatt agcggatcat tatcaacaaa acacgccgat tggtgacggg
1081 cctgtactgt tacctgacaa ccactacctg agcacccagt cagcactgag caaagatccg
1141 aacgaaaaac gcgatcacat ggttctgtta gaattcgtga ccgctgcagg cattactcac
1201 ggaatggacg aactctacaa gtaataatga ggtaccctcg agtctggtaa agaaaccgct
1261 gctgcgaaat ttgaacgcca gcacatggac tcgtctacta gcgcagctta attaacctag
1321 gctgctgcca ccgctgagca ataactagca taaccccttg gggcctctaa acgggtcttg
1381 aggggttttt tgctgaaagg aggaactata tccggattgg cgaatgggac gcgccctgta
1441 gcggcgcatt aagcgcggcg ggtgtggtgg ttacgcgcag cgtgaccgct acacttgcca
1501 gcgccctagc gcccgctcct ttcgctttct tcccttcctt tctcgccacg ttcgccggct
1561 ttccccgtca agctctaaat cgggggctcc ctttagggtt ccgatttagt gctttacggc
1621 acctcgaccc caaaaaactt gattagggtg atggttcacg tagtgggcca tcgccctgat
1681 agacggtttt tcgccctttg acgttggagt ccacgttctt taatagtgga ctcttgttcc
1741 aaactggaac aacactcaac cctatctcgg tctattcttt tgatttataa gggattttgc
1801 cgatttcggc ctattggtta aaaaatgagc tgatttaaca aaaatttaac gcgaatttta
1861 acaaaatatt aacgtttaca atttctggcg gcacgatggc atgagattat caaaaaggat
1921 cttcacctag atccttttaa attaaaaatg aagttttaaa tcaatctaaa gtatatatga
1981 gtaaacttgg tctgacagtt accaatgctt aatcagtgag gcacctatct cagcgatctg
2041 tctatttcgt tcatccatag ttgcctgact ccccgtcgtg tagataacta cgatacggga
2101 gggcttacca tctggcccca gtgctgcaat gataccgcga gacccacgct caccggctcc
2161 agatttatca gcaataaacc agccagccgg aagggccgag cgcagaagtg gtcctgcaac
2221 tttatccgcc tccatccagt ctattaattg ttgccgggaa gctagagtaa gtagttcgcc
2281 agttaatagt ttgcgcaacg ttgttgccat tgctacaggc atcgtggtgt cacgctcgtc
2341 gtttggtatg gcttcattca gctccggttc ccaacgatca aggcgagtta catgatcccc
2401 catgttgtgc aaaaaagcgg ttagctcctt cggtcctccg atcgttgtca gaagtaagtt
2461 ggccgcagtg ttatcactca tggttatggc agcactgcat aattctctta ctgtcatgcc
2521 atccgtaaga tgcttttctg tgactggtga gtactcaacc aagtcattct gagaatagtg
2581 tatgcggcga ccgagttgct cttgcccggc gtcaatacgg gataataccg cgccacatag
2641 cagaacttta aaagtgctca tcattggaaa acgttcttcg gggcgaaaac tctcaaggat
2701 cttaccgctg ttgagatcca gttcgatgta acccactcgt gcacccaact gatcttcagc
2761 atcttttact ttcaccagcg tttctgggtg agcaaaaaca ggaaggcaaa atgccgcaaa
2821 aaagggaata agggcgacac ggaaatgttg aatactcata ctcttccttt ttcaatcatg
2881 attgaagcat ttatcagggt tattgtctca tgagcggata catatttgaa tgtatttaga
2941 aaaataaaca aataggtcat gaccaaaatc ccttaacgtg agttttcgtt ccactgagcg
3001 tcagaccccg tagaaaagat caaaggatct tcttgagatc ctttttttct gcgcgtaatc
3061 tgctgcttgc aaacaaaaaa accaccgcta ccagcggtgg tttgtttgcc ggatcaagag
3121 ctaccaactc tttttccgaa ggtaactggc ttcagcagag cgcagatacc aaatactgtc
3181 cttctagtgt agccgtagtt aggccaccac ttcaagaact ctgtagcacc gcctacatac
3241 ctcgctctgc taatcctgtt accagtggct gctgccagtg gcgataagtc gtgtcttacc
3301 gggttggact caagacgata gttaccggat aaggcgcagc ggtcgggctg aacggggggt
3361 tcgtgcacac agcccagctt ggagcgaacg acctacaccg aactgagata cctacagcgt
3421 gagctatgag aaagcgccac gcttcccgaa gggagaaagg cggacaggta tccggtaagc
3481 ggcagggtcg gaacaggaga gcgcacgagg gagcttccag ggggaaacgc ctggtatctt
3541 tatagtcctg tcgggtttcg ccacctctga cttgagcgtc gatttttgtg atgctcgtca
3601 ggggggcgga gcctatggaa aaacgccagc aacgcggcct ttttacggtt cctggccttt
3661 tgctggcctt ttgctcacat gttctttcct gcgttatccc ctgattctgt ggataaccgt
3721 attaccgcct ttgagtgagc tgataccgct cgccgcagcc gaacgaccga gcgcagcgag
3781 tcagtgagcg aggaagcgga agagcgcctg atgcggtatt ttctccttac gcatctgtgc
3841 ggtatttcac accgcatata tggtgcactc tcagtacaat ctgctctgat gccgcatagt
3901 taagccagta tacactccgc tatcgctacg tgactgggtc atggctgcgc cccgacaccc
3961 gccaacaccc gctgacgcgc cctgacgggc ttgtctgctc ccggcatccg cttacagaca
4021 agctgtgacc gtctccggga gctgcatgtg tcagaggttt tcaccgtcat caccgaaacg
4081 cgcgaggcag ctgcggtaaa gctcatcagc gtggtcgtga agcgattcac agatgtctgc
4141 ctgttcatcc gcgtccagct cgttgagttt ctccagaagc gttaatgtct ggcttctgat
4201 aaagcgggcc atgttaaggg cggttttttc ctgtttggtc actgatgcct ccgtgtaagg
4261 gggatttctg ttcatggggg taatgatacc gatgaaacga gagaggatgc tcacgatacg
4321 ggttactgat gatgaacatg cccggttact ggaacgttgt gagggtaaac aactggcggt
4381 atggatgcgg cgggaccaga gaaaaatcac tcagggtcaa tgccagcgct tcgttaatac
4441 agatgtaggt gttccacagg gtagccagca gcatcctgcg atgcagatcc ggaacataat
4501 ggtgcagggc gctgacttcc gcgtttccag actttacgaa acacggaaac cgaagaccat
4561 tcatgttgtt gctcaggtcg cagacgtttt gcagcagcag tcgcttcacg ttcgctcgcg
4621 tatcggtgat tcattctgct aaccagtaag gcaaccccgc cagcctagcc gggtcctcaa
4681 cgacaggagc acgatcatgc tagtcatgcc ccgcgcccac cggaaggagc tgactgggtt
4741 gaaggctctc aagggcatcg gtcgagatcc cggtgcctaa tgagtgagct aacttacatt
4801 aattgcgttg cgc
//
```

1. A method of selecting a candidate enzyme variant from a library of enzyme variants for the production of a metabolite comprising
providing a plurality of first nucleotide sequences each encoding a different enzyme variant of the library,
providing a precursor molecule wherein the enzyme variant when expressed converts the precursor molecule to the metabolite,
providing a second nucleotide sequence encoding a sensor biomolecule,
providing a third nucleotide sequence encoding a reporter,
wherein the sensor biomolecule when expressed interacts with the metabolite and induces the expression of the reporter in a manner dependent on the concentration of the produced metabolite, and
screening the enzyme variants by detecting the reporter to identify the candidate enzyme variant.

2. The method of claim 1 wherein the enzyme variant converts the precursor molecule to the metabolite directly or through one or more intermediate steps.

3. The method of claim 2 wherein one or more of the intermediate steps are completely or partially randomized.

4. The method of claim 1 wherein the first, second or third nucleotide sequence is DNA or RNA.

5. The method of claim 4 wherein the DNA and/or RNA is linear or included on a plasmid.

6. The method of claim 1 wherein the nucleotide sequences can be physically separated or attached or any combination thereof.

7. The method of claim 1 wherein cofactors are further provided.

8. The method of claim 1 wherein the enzyme variants, the sensor biomolecule and the reporter are produced using a cell-free expression system.

9. The method of claim 1 wherein the enzyme variants, the sensor biomolecule and the reporter can be produced directly in an evaluation vessel.

10. The method of claim 9 wherein the evaluation vessel is in an emulsion or microtiter well format.

11. The method of claim 1 wherein the enzyme variants, the sensor biomolecule and the reporter can be produced outside and then combined in an evaluation vessel.

12. The method of claim 8 wherein the cell-free expression system comprising commercially available in vitro translation reagents and/or kits.

13. The method of claim 1 wherein the candidate enzyme variant is validated by sequencing the nucleotide encoding the enzyme variant.

14. The method of claim 1 wherein enzyme variants and/or sensor biomolecules are provided.

15. The method of claim 1 wherein the selection process is repeated on a subset of identified candidate enzyme variants for optimization.

16. The method of claim 1 wherein the reporter is a fluorescent protein.

17. The method of claim 16 wherein the fluorescent protein is GFP.

18. The method of claim 1 wherein the reporter is a member selected from the group consisting of mPlum, mCherry, tdTomato, mStrawberry, J-Red, DsRed-monomer, mOrange, mKO, mCitrine, Venus, YPet, EYFP, Emerald, EGFP, CyPet, mCFPm, Cerulean, T-Sapphire, Firefly (FLuc), modified firefly (Ultra-Clo), Click beetle (CBLuc), Sea pansy (RLuc), Copepod crustacean (GLuc), and Ostracod crustacean (CLuc).

19. The method of claim 1 wherein the reporter further comprises luciferase for detection by light, pigments for detection by color, surfactants for detection by emulsion breaking, and adhesives for detection by adhesion.

20. The method of claim 1 wherein the screening is carried out by fluorescent microscopy, microtiter plate assay, emulsion assay, microfluidic assay, pull-down assay or luciferase high throughput screening.

21. The method of claim 1 wherein the sensor biomolecule and the metabolite binding partner is a member pair selected from the group consisting of AcuR/acrylate, cdaR/glucaric acid, ttgR/naringennin, ttgR/phenol, btuB riboswitch/cobalamin, mphR/macrolides, tetR/tetracycline derivates, benM/muconic acid, alkS/medium chain n-alkanes, xylR/xylose, araC/Arabinose, gntR/Gluconate, galS/Galactose, trpR/tryptophan, qacR/Berberine, rmrR/Phytoalexin, cymR/Cumate, melR/Melibiose, rafR/Raffinose, nahR/Salicylate, nocR/Nopaline, clcR/Chlorobenzoate, varR/Virginiamycin, rhaR/Rhamnose, PhoR/Phosphate, MalK/Malate, GlnK/Glutamine, Retinoic acid receptor/Retinoic acid, LacI/allolactose, Estrogen receptor/Estrogen and Ecdysone receptor/Ecdysone.

22. The method of claim 1 wherein the sensor biomolecule is a transcription factor, riboswitch, two-component signaling protein, a nuclear hormone receptor, a G-protein coupled receptor, a periplasmic binding protein, or an engineered protein switch.

23. The method of claim 1 wherein the sensor biomolecule is cdaR and the metabolite is a diacid.

24. The method of claim 22, wherein the biosensor is an engineered protein switch such as an engineered calmodulin.

25. The method of claim 1 wherein the sensor is AcuR and the metabolite is acrylate.

26. The method of claim 1 wherein the enzyme is PCS, MIOX, Udh, or INO1.

27. The method of claim 1 wherein the precursor molecule is 3-hydroxypropionate.

28. The method of claim 1 wherein the reporter protein is an emulsion-breaking protein.

29. The method of claim 1 wherein the plurality of first nucleotide sequences encoding the different enzyme variants are generated by methods comprising gene synthesis, error prone PCR, targeted mutagenesis, or oligonucleotide directed mutagenesis.

30. A method of identifying a candidate sensor biomolecule variant from a library of sensor biomolecule variants for a metabolite comprising
providing a plurality of first nucleotide sequences each encoding a different sensor biomolecule variant of the library of sensor biomolecule variants,
providing a metabolite,
providing a second nucleotide sequence encoding a reporter,
wherein the sensor biomolecule variant when expressed interacts with the metabolite and induces the expression of the reporter in a manner dependent on the concentration of the produced metabolite, and
screening the sensor biomolecule variants by detecting the reporter to identify the candidate sensor biomolecule variant.

31. A cell-free bio-sensing system for selecting a candidate enzyme variant from a library of enzyme variants for the production of a metabolite comprising:
a plurality of first nucleotide sequences each encoding a different enzyme variant of the library of enzyme variants,
a precursor molecule wherein the enzyme variant when expressed converts the precursor molecule to the metabolite,
a second nucleotide sequence encoding a sensor biomolecule,
a third nucleotide sequence encoding a reporter,
wherein the sensor biomolecule when expressed interacts with the metabolite and induces the expression of the reporter in a manner dependent on the concentration of the produced metabolite, and
wherein the enzyme variants are screened by detecting the reporter to identify the candidate enzyme variant.

32. The system of claim 31 wherein the enzyme variants convert the precursor molecule to the metabolite directly or through one or more intermediate steps.

33. The system of claim 32 wherein one or more of the one or more intermediate steps are completely or partially randomized.

34. The system of claim 31 wherein the first, second or third nucleotide sequence is DNA or RNA.

35. The system of claim 34 wherein the DNA and/or RNA is linear or included on a plasmid.

36. The system of claim 31 wherein the nucleotide sequences can be physically separated or attached or any combination thereof.

37. The system of claim 31 further comprises cofactors.

38. The system of claim 31 wherein the enzyme variants, the sensor biomolecule and the reporter are produced using a cell-free expression system.

39. The system of claim 31 wherein the enzyme variants, the sensor biomolecule and the reporter can be produced directly in an evaluation vessel.

40. The system of claim 39 wherein the evaluation vessel is in an emulsion or microtiter well format.

41. The system of claim 31 wherein the enzyme variants, the sensor biomolecule and the reporter can be produced outside and then combined in an evaluation vessel.

42. The system of claim 38 wherein the cell-free expression system comprising commercially available in vitro translation reagents and/or kits.

43. The system of claim 31 wherein the candidate enzyme variant is validated by sequencing the nucleotide encoding the enzyme variant.

44. The system of claim 31 wherein enzyme variants and/or sensor biomolecules are provided.

45. The system of claim 31 wherein the selection process is repeated on a subset of identified candidate enzyme variants for optimization.

46. The system of claim 31 wherein the reporter is a fluorescent protein.

47. The system of claim 46 wherein the fluorescent protein is GFP.

48. The system of claim 31 wherein the reporter is a member selected from the group consisting of mPlum, mCherry, tdTomato, mStrawberry, J-Red, DsRed-monomer, mOrange, mKO, mCitrine, Venus, YPet, EYFP, Emerald, EGFP, CyPet, mCFPm, Cerulean, T-Sapphire, Firefly (FLuc), modified firefly (Ultra-Clo), Click beetle (CBLuc), Sea pansy (RLuc), Copepod crustacean (GLuc), and Ostracod crustacean (CLuc).

49. The system of claim 31 wherein the reporter further comprises luciferase for detection by light, pigments for detection by color, surfactants for detection by emulsion breaking, and adhesives for detection by adhesion.

50. The system of claim 31 wherein the screening is carried out by fluorescent microscopy, microtiter plate assay, emulsion assay, microfluidic assay, pull-down assay or luciferase high throughput screening.

51. The system of claim 31 wherein the sensor biomolecule and the metabolite binding partner is a member pair selected from the group consisting of AcuR/acrylate, cdaR/glucaric acid, ttgR/naringennin, ttgR/phenol, btuB riboswitch/cobalamin, mphR/macrolides, tetR/tetracycline derivates, benM/muconic acid, alkS/medium chain n-alkanes, xylR/xylose, araC/Arabinose, gntR/Gluconate, galS/Galactose, trpR/tryptophan, qacR/Berberine, rmrR/Phytoalexin, cymR/Cumate, melR/Melibiose, rafR/Raffinose, nahR/Salicylate, nocR/Nopaline, clcR/Chlorobenzoate, varR/Virginiamycin, rhaR/Rhamnose, PhoR/Phosphate, MalK/Malate, GlnK/Glutamine, Retinoic acid receptor/Retinoic acid, LacI/allolactose, Estrogen receptor/Estrogen and Ecdysone receptor/Ecdysone.

52. The system of claim 31 wherein the sensor biomolecule is a transcription factor, riboswitch, two-component signaling protein, a nuclear hormone receptor, a G-protein coupled receptor, a periplasmic binding protein, or an engineered protein switch.

53. The system of claim 31 wherein the sensor biomolecule is cdaR and the metabolite is a diacid.

54. The system of claim 53, wherein the biosensor is an engineered protein switch such as an engineered calmodulin.

55. The system of claim 31 wherein the sensor is AcuR and the metabolite is acrylate.

56. The system of claim 31 wherein the enzyme is PCS, MIOX, Udh, or INO1.

57. The system of claim 31 wherein the precursor molecule is 3-hydroxypropionate.

58. The system of claim 31 wherein the reporter protein is an emulsion-breaking protein.

59. The system of claim 31 wherein the plurality of the first nucleotide sequences encoding the different enzyme variants are generated by methods comprising gene synthesis, error prone PCR, targeted mutagenesis, or oligonucleotide directed mutagenesis.

60. A cell-free bio-sensing system for identifying a candidate sensor biomolecule variant from a library of sensor biomolecule variants for a metabolite comprising
a plurality of first nucleotide sequences each encoding a different sensor biomolecule variant of the library of sensor biomolecule variants,
a metabolite,
a second nucleotide sequence encoding a reporter,
wherein the sensor biomolecule variant when expressed interacts with the metabolite and induces the expression of the reporter in a manner dependent on the concentration of the produced metabolite, and
wherein the sensor biomolecule variants are screened by detecting the reporter to identify the candidate sensor biomolecule variant.

61. The method of claim 1 wherein the enzyme variants or the first nucleotide sequences encoding the enzyme variants are attached to a solid support for multiplex screening of candidate enzyme variants.

62. The method of claim 61 wherein the solid support comprises multiple compartments in membrane, filter, paper, gel, plate, slide format and the like.

63. The method of claim 62 wherein an individual enzyme variant or an individual nucleotide sequence encoding the enzyme variant is trapped in an individual compartment of the multi-compartment solid support.

64. The method of claim 63 wherein the enzyme variant is isolated with corresponding precursor molecules and reporter sequences inside an individual compartment.

65. The method of claim 63 wherein each individual compartment is immobilized, or temporarily immobilized, within the multi-compartment solid support.

66. The method of claim 63 wherein the individual compartment can be sorted by an automated sorting system.

67. The method of claim 63 wherein the individual compartment can be separated from the multi-compartment solid support by manual extraction.

68. The method of claim 63 wherein the candidate enzyme variant can be identified based on the known content of each individual compartment, or by targeted sequencing, or in-situ imaging.
